(19) United States
(12) Patent Application Publication
(10) Pub. No.: US 2026/0034152 A1
(43) Pub. Date: Feb. 5, 2026

(54) Influx Vitamins and Drinks, with Zinc Ionophoric Influx

(71) Applicant: Stephen Black, Stafford, VA (US)

(72) Inventor: Stephen Black, Stafford, VA (US)

(21) Appl. No.: 18/307,684

(22) Filed: Apr. 26, 2023

(51) Int. Cl. A61K 31/7004; A61K 31/315; A61K 31/352; A61K 31/375; A61K 31/593; A61K 31/737; A61K 33/06; A61K 33/34

(52) U.S. Cl. CPC A61K 31/7004; A61K 31/315; A61K 31/352; A61K 31/375; A61K 31/593; A61K 31/737; A61K 33/06; A61K 33/34

(57) ABSTRACT

I am a practicing physician who has not only recommended these same vitamin ingredients to roughly over 2000 patients. I have also monitored the progress long with other health recommendations such as exercise, proper diet, rest, and spinal adjustments. I have achieved with the patient amazing results of immune support and a reduction of inflammation (reducing inflammation reduces pain) in an inhospitable environment such as today. I am perhaps alone in my knowledge and patient experience and I wish to share these achievements with the world (companies will copy my vitamin volume ideas). However, I must first patent my results of my many healthy patients.

### BACKGROUND

### Field of the Invention: Immune Support with Significant Reduction of Inflammation

My Inventions are Formulas of Vitamins and Immune Drinks to Support the Immune System and to Reduce Inflammation.

Example: Formula 5 Quercetin, Zinc, D3, Calcium, Sodium, and Potassium Immune Drink
1-5000 mg Quercetin in any variation in a drink form including Quercetin Syrup Diluted with fluid to make an Immune drink or refreshment. 250 mg used in this formula
1-200 mg of Zinc in any variation. 25 mg used in this formula
1-200 mcg of vitamin D3 of any variation. 25 mcg used in this formula
1-2000 mg of Calcium of any variation. 200 mg used in this formula
1-500 mg of Sodium Ions such as in an energy drink (160 mg per 16 oz. is commonly used and is what I use)
1-300 mg of Potassium Ions such as in energy drinks (45 mg per 16 oz. is commonly used and is what I use)
16-32 oz container is best

This Formula is best for reducing Inflammation and Immune Support.

### Description of the Related Art

There are 1-2 Vitamin combinations on the market with quercetin and zinc only. There are also 1-2 vitamin combinations with Elderberry and zinc combinations. I do not know if they have a patent. These are deficient in there milligram amounts for reducing inflammation and immune support. The more ingredients the product has the more deficient they are without a real beneficial stimulation in the human body. In other words their daily recommendations are deficient. This field of vitamins in the immune support market is growing fast with the current environment we live in. However, truly there are few decent products and none have a real knowledge of clinical use. There are few products similar in that they don't have clinical studies and no real knowledge of how much and what ingredients are required per day.

### BRIEF DESCRIPTION OF THE DIAGRAMS

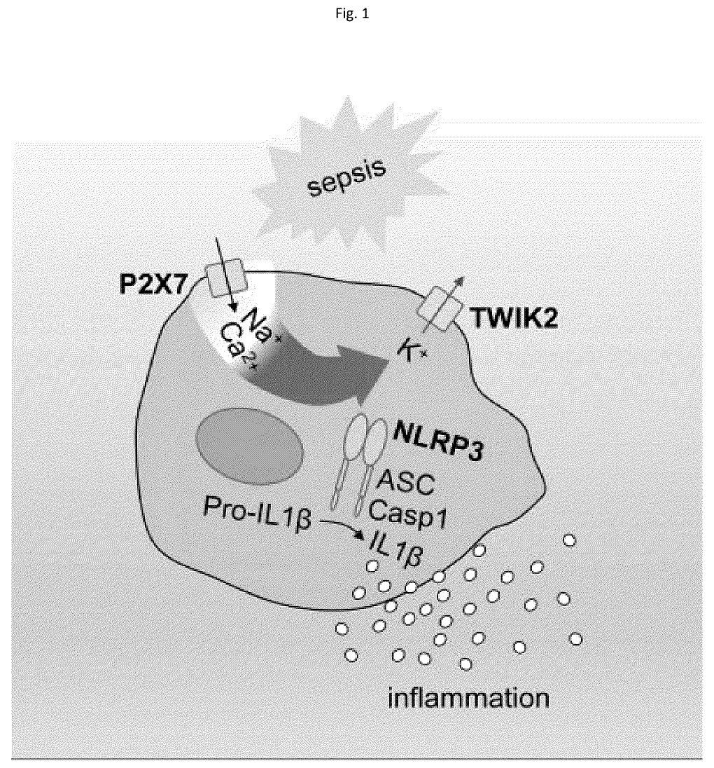

FIG. 1

I am including this diagram to describe the cellular action discovered by Dr. Jalees Rehman and Professor Asrar Malik of the University of Illinois in Chicago.

This diagram is a diagram of a macrophage white blood cell. This describes how White blood cells become overtly hyper excitatory with potassium and TWIK2 potassium channels. Then release Interluekin-1 Beta. Interluekin-1 Beta is associated with increasing Fibroblasts, Collagenase, and Cortisol. Inhibiting newly discovered Twik2 channels does in fact inhibit inflammation. Dr. Jalees Rehman and Professor Asrar Malik discovered Quinine can inhibit TWIK2 channels.

This can also be very effective in shutting down overly excited white blood cells in what has recently been called a cytokine response or storm. This is where white blood cells attack your own body because of over stimulation. Shutting down the Twik2 channels can shut down cytokine response of the compliment system. The compliment system is when a WBC punches a very tiny hole in an organ or system.

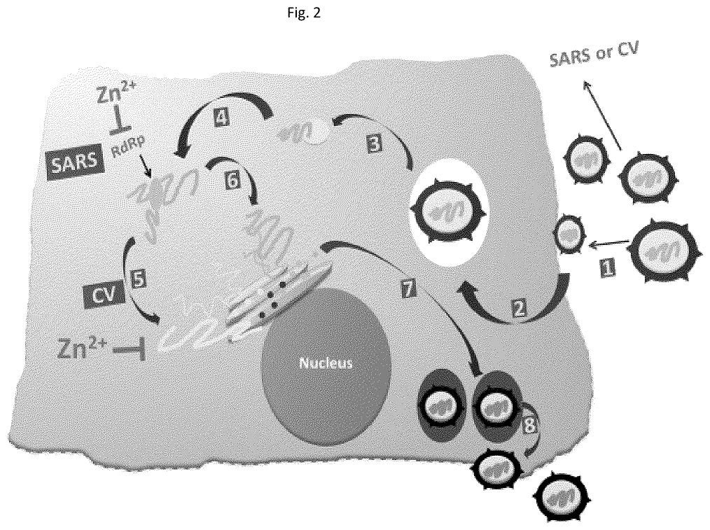

FIG. 2

This is a diagram of Zinc Ionophore Influx across the human cell plasma membrane. Plant polyphenols such as flavonoids like quercetin and Eppigallocatecin-gallate act as antioxidants and also carry zinc ionophore across the cell membrane. This makes the zinc a patient might take many times more effective by getting zinc inside the cell.

This action occurs when combining Quinine and zinc as well as Quercetin (or Eppigallocatecin-gallate) and zinc as well as Elderberry and Zinc. This action occurs also in other fruits and vegetables (berries, cherries, grapes, red onions, baby kale, spinach, and cherry tomatoes ect . . . ) with high concentrations of the bioflavonoid Quercetin. Quercetin is the active bioflavonoid in elderberry. There are other fruits and vegetables (berries, cherries, grapes, red onions, baby kale, spinach, and cherry tomatoes ect . . . ) with high concentrations of the bioflavonoid Quercetin.

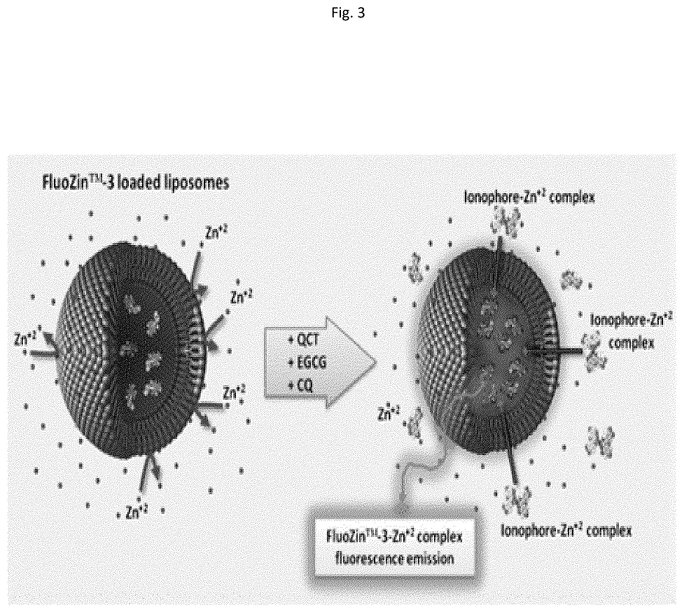

FIG. 3

This is a diagram of How Zinc inhibits the replication of viruses that try to penetrate the cell wall and cause cell death.

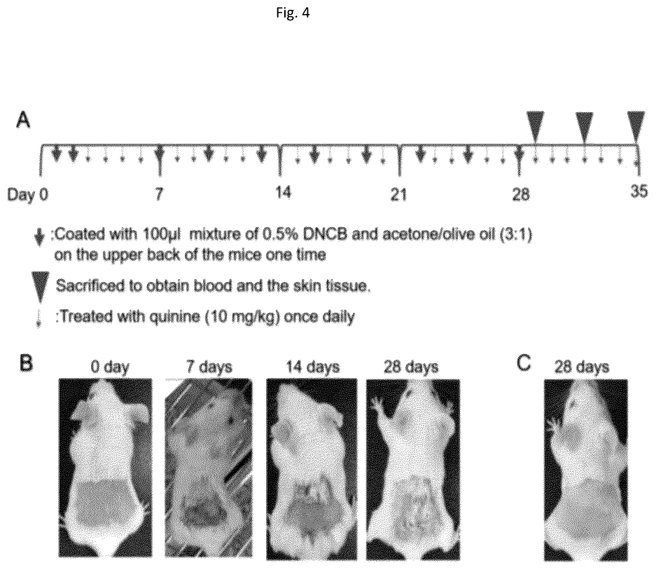

FIG. 4

This is a diagram of a study showing the anti-inflammatory properties of quinine on mice. Acetone/olive oil was placed on the mice for 4 weeks. Mice given quinine did not have a loss of skin and flesh due to the suppression of TAS2R channels in the lungs inhibited by the quinine. The inhibited TAS2R receptors cause an agonist of IGE immunoglobulin. IGE cells are associates with allergic reactions in the human body. Reducing allergic reactions in the body will inhibit inflammation. This is a great illustration of reduction of skin inflammation.

The main idea is to get fruit and vegetable concentrate extract (powder concentrate extract, syrup concentrate extract, fluid concentrate extract) with high volume of Quercetin to open up the human cell plasma membrane for a zinc ionophoric influx reaction. Then add sufficient Zinc to enhance the patient's immune system.

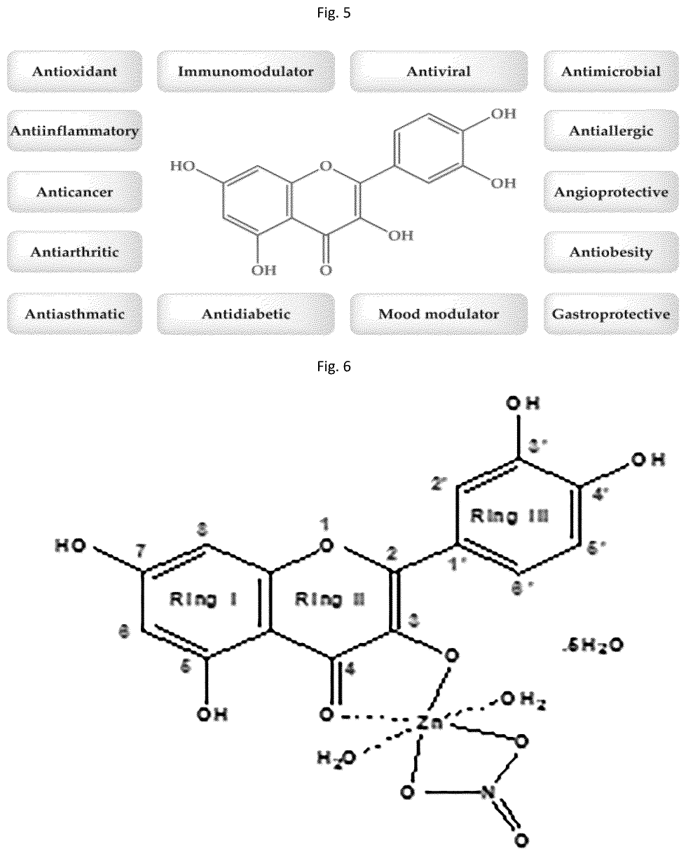

FIG. 5

This is a unique diagram of Quercetin and all the helpful body functions Quercetin will influence in a patient who takes the flavonoid daily. I have recommended to all my patients since the beginning of the covid-19 pandemic to take zinc with Quercetin to enhance the positive biological effects. The effects are antioxidant, anti-inflammatory, anticancer, anti-arthritic, anti-asthmatic, anti-diabetic, antiviral, antimicrobial, anti-allergic, angioprotective, anti-obesity, gastro protective, and mood modulator. The anti-inflammatory aspects of Quercetin are derived from its inhibition of Interleukin beta, interleukin 6, and interleukin 8 signals released from white blood cells (Macrophages). Anti-allergic response is derived from Quercetin's ability to inhibit eosinophil activity. The anticancer ability of Quercetin is achieved through deletion of free radicals intra cellular.

FIG. 6

This is a diagram of the carbon compound of Quercetin joining with zinc through chelating. Zinc combines to Quercetin on its second carbon non aromatic ring (between its 1 and 3 aromatic carbon ring). Quercetin and Zinc a chelating ligand together through carbonyl C (4) =o oxygen and phenolic C (3)-OH oxygen. Quercetin and Zinc have a distorted octahedral geometry that alleviates hyperglycemia. Zinc heals Pancreatic Beta cells and this function is accelerated with Quercetin. Quercetin improves antioxidant potential and prevents oxidative damage.

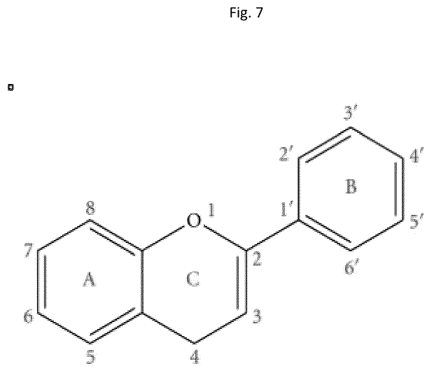

FIG. 7

However there are other pathways to get zinc Ionophoric influx reaction within the cell membrane. The other pathways require to get fruit and vegetable concentrate extract (powder concentrate extract, syrup concentrate extract, fluid concentrate extract) with high volume of Quercetin or other flavonoids to open up the human cell plasma membrane for a zinc ionophoric influx reaction. Then add sufficient Zinc to enhance the patient's immune system. Other flavonoids have a flavonoid carbon skeletal structure. One Aromatic Ring is connected to a second Aromatic Ring by a carbon bridge, which consists of 3 carbon Atoms. These are also known as an Aromatic Polyketide flavonoid group. This forms a cyclic structure as illustrated in FIG. 7.

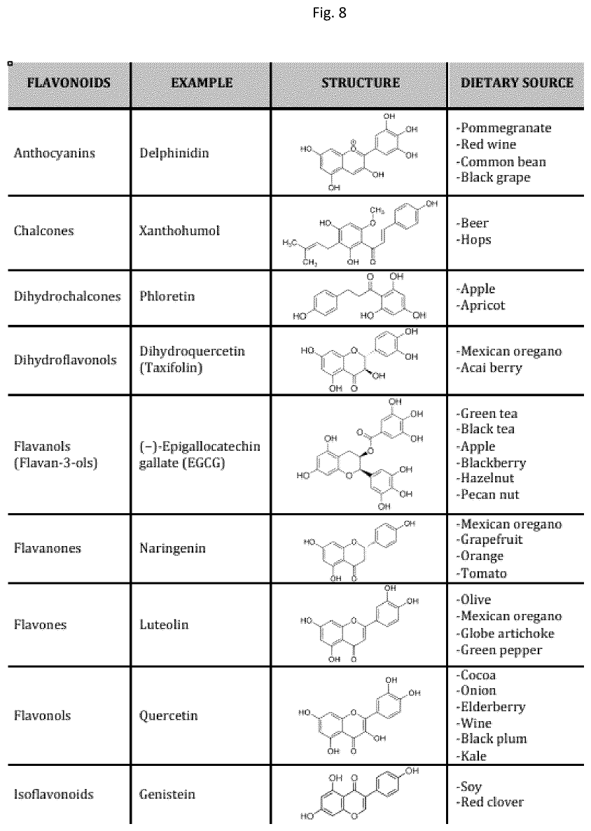

FIG. 8

This is a perfect diagram of that shows flavonoids, an example of a flavonoid, the 3 carbon structure, and in what foods they are found. This will get us started with a list of other pathways to zinc ionophic influx. The first is Anthocyanines, Delphinidin, then is Chalcones, xanthohumol, then Dihydrochalones, Phloretin, then Dihydidroflavonols, Dihydroquercetin (acai berry), then Flavanols (FLavan-3-ols), Epigallatechin Gallate, then Flavanones, Nerigenine, Flavones, Luteolin, then Flavonols Quercetin, then Isoflavanoids, Genistein. Some of these flavonoids have better zinc ionophoric activity than others. We will discuss and list compounds and ingredients that are proven zinc ionophoric influx (Quercetin, Elderberry, Quinine, ect . . . ) and list others that or not as effective or have yet to be fully studied and proven. Phloritin can inhibit UGT that processes bilirubin when too much is consumed. This entire diagram does depict compounds that all reduce inflammation very effectively. Naringenine, Xanthohumol, Phloritin, and Dihydrochalones are all excellent metal chelators. The easiest metals to chelate are Iron (FE) and Copper (CU). All metal chelators will have some affinity to Chelate Zinc between 5%-100%. Naringenine and Xanthohumol will have been known to chelate zinc as well. This particular diagram has a study that does prove Quercetin, Dihyroquercetin, Luteolin, Epigallatechin Gaallate, Pyrithione, Tannic acid, and Gallic Acid to be effective in Zinc ionophoric Influx.

FIG. 9

This is an amazing study of zinc chelating in solution (left) and zinc Liposome assay (right) comparison. First we will describe the chelating strength of zinc cations by polyphenols, pryothione, clioquinol, and TPEN in sodium. The quenching of zinc dependent fluorescence of Fluozin-3 indicates capacity of compounds at 10 (micro) M to retrieve zinc cations from zinc-Fluozin-3 complex formed between 3 (micro) M Fluozin-3 and 1 (micro) M Zncl2. Inset 3 (micro) M Fluozin-3 calibration plot 494 nm? 516 nm. All values are means ISD of 3 independent experiments.

Second we will describe zinc liposome assay for Zinc ionophoric Influx. Liposome Assay for the determination of zinc ionophore activity of Polyphenols, Clioquinol, and Pryothione. The increase of zinc dependent fluorescence of Fluozin-3 indicates the capacity of the compounds at 50 (micro) M to interact with 10 (micro) M zinc cations. Transport them across the liposome membrane and present them to 3 UM Fluozin-3 in liposome inner cavity. Control are Fluozin-3 loaded liposome in the presence of 10 (micro) M zinc cations with 0.1% DMSO. Fluozin-3 loaded liposomes in the absence of zinc (lipo-Fz3) and in the presence of 10 UM zncl2 (Zn 10). 494 nm/516 nm. All values are means ISD of three independent experiments.

This study is very important as an experiment to measure zinc chelation (the capacity of compounds to retrieve zinc cations) and zinc ionophoric influx across the cell membrane (liposomes in the inner cell cavity). Ultimately this is a system that I believe is accurately defining what to use in a clinical study for zinc ionophoric influx. It is not used that often as it should be. The results were in order with highest zinc ionophoric influx Clinoquinol (synthetic not a compound of my ingredients), Pyrothione, Epigallocatechin, Quercetin, Luteolin, Tannic Acid, Gallic Acid, Rutin, Caffeic Acid, Catechol, Taxifolin, catechin (Cat Hyd), Genestein, Resveratol, Phloratin, and Naringinin (lowest zinc ionophoric influx). Clinoquinol is a synthetic version of the alkaloid quinine. This would indicate why quinine is a huge zinc ionophoric influx ingredient. All of these have effective zinc chelation and either an abundance of zinc ionophoric influx, adequate zinc ionophoric influx or mildly some zinc ionophoric influx. Epigallocatechin, Quercetin, Luteolin, Tannic Acid, Gallic Acid, Caffeic Acid, Catechol, Taxifolin, and Catechin are widely used in studies as zinc ionophoric influx for zinc to cross the cell membrane. Pyrothione has potential as skin therapy mostly. Some studies at times denoted zinc depravation due to Pyrothione negative effects on cellular DNA. Zinc depravation causes ROS excess in many studies outside the human anatomy. Zinc becomes cytotoxic in the human body if zinc extracellular concentration exceeds capacity of zinc homeostatic system. This excess causes apoptotic system machine or cell death in laboratory studies outside the body. Epigallocatechin, Quercetin, Luteolin, Tannic Acid, Gallic Acid, Caffeic Acid, Catechol, Taxifolin, and Catechin are widely used in studies as zinc ionophoric influx for zinc to cross the cell membrane. This study is important because it shows nearly all flavonoid compounds have 5%-100% zinc ionophoric activity. Even a lower zinc ionophoric activity can translate into large amounts of zinc crossing the cell membrane. Also I have experienced success with some of these with many of my patients.

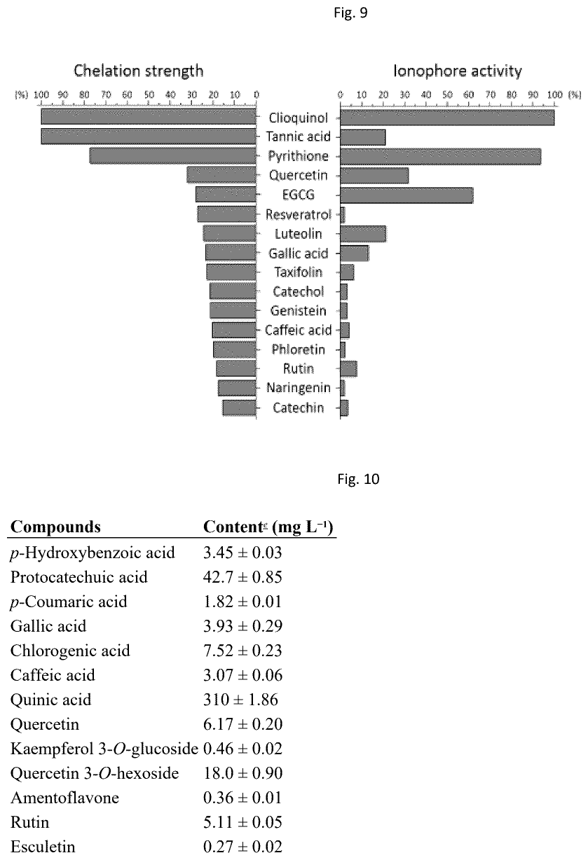

FIG. 10

This is a diagram of Elderberry compounds and the content in (mg L exponent-1). The list includes P-hydroxybenzoic Acid (3.45), Protocatechuic Acid (42.7), P-Coumaric Acid (1.82), Gallic Acid (3.93), CHolorgenic Acid (7.52), Caffeic Acid (3.07), Quinic Acid (310), Quercetin (6.17), Kaempferol-3-o-Glucoside (0.46), Quercetin 3-o-hexide (18), Amentoflavone (0.36), Rutin (5.11), Esculetin (0.27). The main ingredients are Quinic Acid, Protocatechuic Acid, Quercetin (this has zinc ionophoric influx and zinc chelation), Quercetin 3-O-hexide (this has zinc ionophoric influx and zinc chelate), and Rutin (this has mild to moderate zinc ionophoric influx and moderate zinc chelate). All these ingredients have a moderate to strong Zinc Chelation. However, only Quercetin, Quercetin-3-O-Hexide, Kaemferol-3-O-Glucoside, and Gallic Acid have a strong zinc ionophric influx across the cell membrane and a strong zinc Chelation. Quininc Acid (cats claw) is the largest ingredient in elderberry. Quinic Acid Inhibits oral anti-inflammatory, (inhibits inflammation of lung type 2 alveolar epithelial cells) properties by inhibiting transcription Nuclear Factor Kappa Beta (NF-kB). Acetylation and added hydroxyl groups weaken the anti-inflammatory response. Quinic acid with added Amide will strengthen the anti-inflammatory response. Quinic Acid also has anti-viral properties and is used for STD's including HIV and the flu. Quinic acid is also related to quinine and gives tonic water its bitter taste.

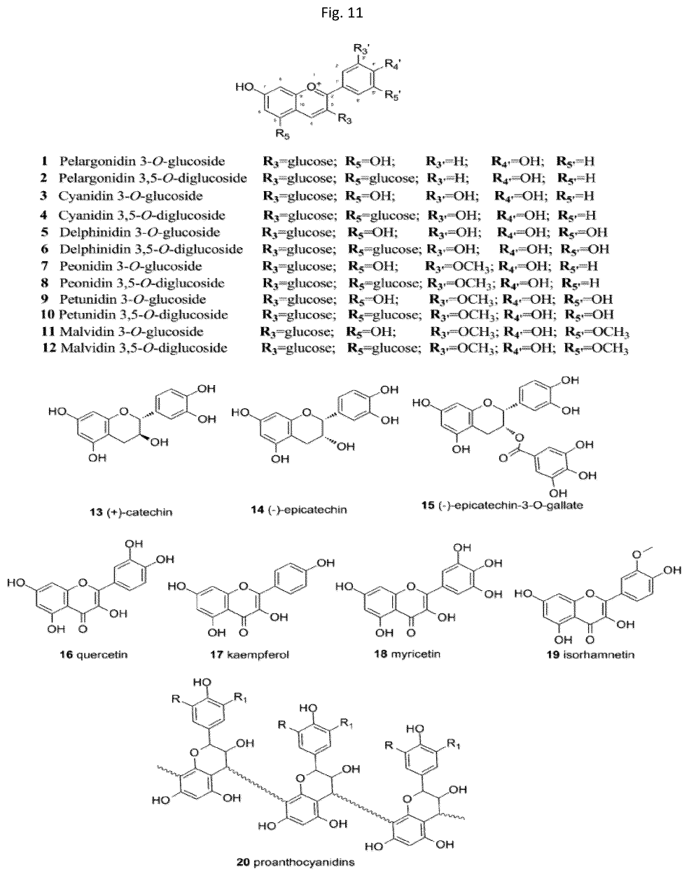

FIG. 11

This is a list most of all the grape flavonoid structures and flavonoid compounds. Pelargonidin 3-O-glucoside, Pelargonidin 3, 5-O-diglucoside, cyaniding 3-O-glucoside, cyaniding 3, 5-Odiglucoside, Delphinidin 3-O-glucoside, Delphinidien 3, 5-O diglucoside, Peonidin 3-O-glucoside, Peonidin 3, 5-O-diglucoside, petunidin 3-O-glucoside, Petunidin 3,5-O diglucoside, Malvidin 3-O-glucoside, and Malvidin 3,5-O-diglucoside are a list of grape flavonoid structures called Anthocyanins. The grape flavonoids are Catechin, Epicatechin, Epicatechin-3-O-gallate, Quercetin, Kaempferol, Myricetin, Isorhamnetin, Proanthocyonidins. The Flavanols of this diagram are Catechin, Epicatechin, and Proanthrocyonidins. White grapes have 46%-56% of these three Flavanols and are the most abundant. Red grapes have 13%-30% of these three flavanols. Grape Flavonols are Quercetin, Kaempferol, myricetin, Laricitin, Isorhamnetin, and syringetin and are the second most abundant. Flavonols Quercetin-3-O-glucouronide and Quercetin-3-O-lucoside are the predominant compound in grapes. Dihydroflavonols of grapes are Astilbin, egeletin, and proanthocynidins. Pro anthocynonidins suppress the activator of inflammatory transcription factor NF-kB and reduces inflammation. Anthocynins are found in red grape variety only and mostly in the skin. Berry development is an important correlation between Anthocyanin biosynthesis. Each Grape has a unique set of Anthycynins. The biological activities of flavonoids are determined by glycosylation, type of sugar residues and acyl esterification. Because of Flavonoid aerobic metabolism active cells create toxic byproducts ROS reactive oxygen species. Antioxidents like Flavonoids neutralize oxidative stress (ROS). Flavonoids represent a large group of high antoxident properties with low molecular weight. Flavonoids can act as metal chelators reducing ferric and cupric ions (also chelation of zinc ionophoric influx). Flavonoids can also inhibit enzymes (inhibiting enzymes involved in ROS generation: xanthine oxidase, Protein kinase C, cyclooxygenase, lipoxygenase, glutathione s transferase, microsomal monooxygenase, mitochondrial succinoxidase, and NADH oxidase). Flavonoids also have antioxidant by increasing the uric acid in the plasma. The Flavonoids involved in zinc ionophoric influx in this diagram are Quercetin, Kaempferol, myricetin (laricitin is produced from myricetin and syringetin is derived from Laricitin), catechin, epicatechin, epicatechin-3-0-gallate Isorhamnetin, and Proanthocyonidins. Syringetin is associated with ostoeblastic bone formation. Isorhamnetin (derivative of Quercetin) and Quercetin promote glucose uptake when taken together. Isorhamnetin is in Ginko Biloba and protects cardio-cerebral vessels. Isorhamnetin, laricetin, and Syringetin have zinc chelation properties but it is unknown to what percent. Isorhamnetin probably has a high affinity for zinc ionophoric influx. Dyhydroflavonols Engeletin and astilbin are used for antibacterial and burn or wound healing. Astilbin suppress neuronal nitric oxide and increases serotonin and dopamine. Engeletin and astilbin are not good candidates for zinc ionophoric influx. Anthocynonidins Pelargonidin, cyanidin, Delphinidin, peonidin, petunidin, Malvidin have a high molecular weight that inhibit carcinogenesis. Anthocynadins are a metal chelators do have zinc ionophoric influx capacity due to the compound shape although it is unknown the exact percent of zinc ionophoric influx (lighter weight are sometimes considered better). The Flavonoids involved in zinc ionophoric influx in this diagram are Quercetin, Kaempferol, myricetin, catechin, epicatechin, epicatechin-3-0-gallate and Anthocynonidins (Anthocynonydins need more study to show the percentage of zinc chelation). These listed inhibit viral MRNA replication with reverse transcriptase-polyrease chain reaction. Their constituent polyphenolic compounds are to scavenge free radicals and chelate metals, not only the redox-active transition metals (copper and Iron), but also redox interactive metals, such as zinc. Isorhamnetin indeed has a high affinity for zinc ionophoric influx when studied.

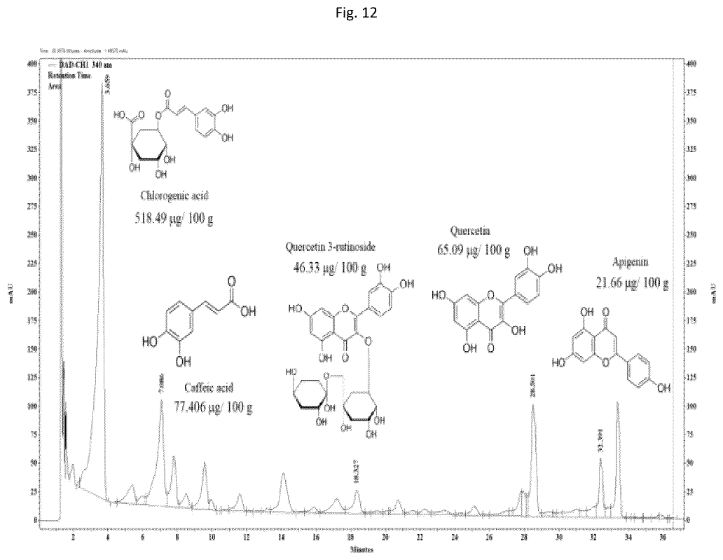

FIG. 12

Quercetin, Quercetin-3-rutinoside and Apigen (flavonoids) occur in abundant quantities in the sour cherry. These are precursor compounds in the biosynthesis of Anthocyanins. There are other Phenolic compounds like chlorogenic Acid and Caffeic Acid in high concentrations in this fruit. The Antioxidant of these compounds is also high. Apigenin is biosynthetically derived from L-tyrosine and L-Phenylalanine then the Shikimate pathway. Apigenin is biogenetically made from tyrosine and phenylalanine. Apigenin is useful as a muscle relaxor. Apigenin can also inhibit angiogenesis, metastasis, Alzheimer's, and Parkinson's. However, Quercetin, Quercetin-3-Rutinose, Apigenin, Chlorogenic Acid, and Caffeic Acid are all Zinc Chelators and have Zinc Ionophoric influx Capabilities. Antiviral activity is connected to non-glycosidic compounds and hydroxylation at the third position.

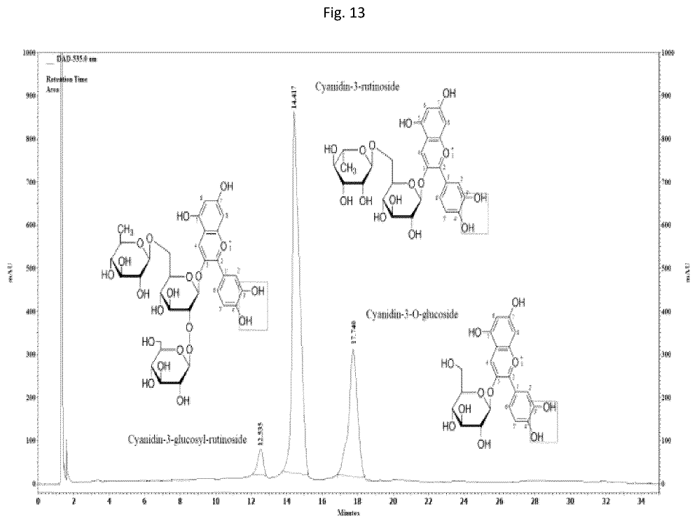

FIG. 13

This is a simple diagram of the largest percent of Amino Acids and Flavonols found in sour Cherries. The main Anthrocyanin components are cyannidin-3-O-glucosyl-rutinside (2 mg/100 mg), cyaniding-3-O-rutinoside (183 mg/100 mg), Cyanidin-3-O-Monoglucoside (4.29 mg/100 mg). The antioxidant capacity of the component is determined by OH groups at the C3 and C4 on the Chalcone. Caroxylic Acids in these sour cherries are Hydroxy Cinnamic Acid (possibly a good zinc chelator), Chlorogenic Acid, Neochlorogenic Acid, and P-Coumaroylquinic Acid. Chlorogenic Acid is an Esters of Caffeic Acid and Quinic Acid. Neochlorogenic acid is an Isomer of Chlorogenic Acid and both Display Zinc Ionophoric Influx capabilities. Flavanols in sour cherries are Catechin, epicatechin, Epigallocatechin, and Gallocatechin. These Flavanols listed are all consistence metal chelators, and are very useful for zinc ionophoric influx (Melatonin is another useful derivative of sour Cherries).

Sweet cherries contain cyonidin-3-O-rutenoside, sour cherries contain cyaniding-3-glucoserutinside. Malic Acid is contained in 1-2% of sour cherries and sweet cherries. The PH for sour cherries is 3.1-3.6 and the PH for sweet is 3.7-4.5. Both sweet and sour contain catechin and epicatechin as Flavanols. Both sweet and sour contain Quercetin and Kaempferol as Flavonols.

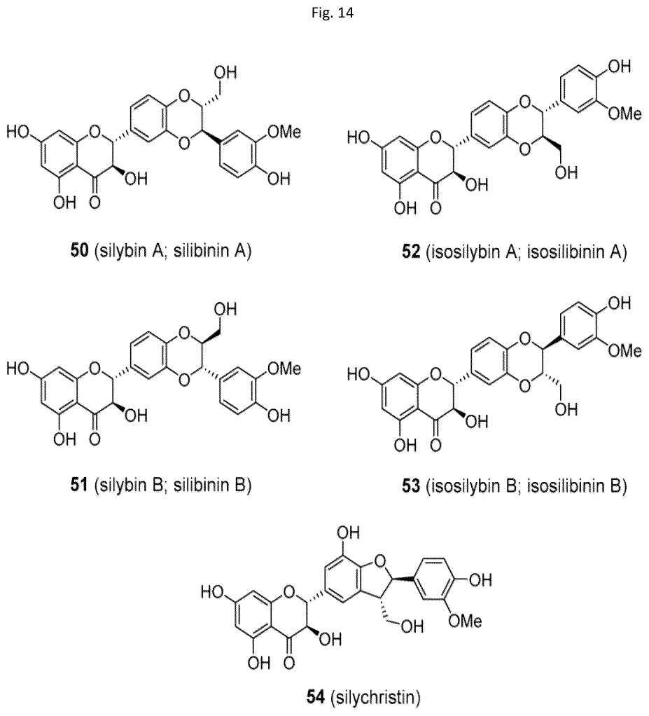

FIG. 14

Silymarin, is a mixture of polyphenolic compounds extracted from Silybum Marianum Gaertin (Asteraceae fruits). Milk Thistle of commerce is a standardized preparation of Silybum Marianum. Asteraceae fruits are well known for hepatoprotective properties. Silybum Is mostly composed of Flavonolignans but also contains Flavonoids (Taxifolin, Quercetin) and Polymeric molecules. The main Flavonolignan components contained in Silymarin are Silybin (AKA Silibin), IsoSilybin, SilyChristin, and Silydianin (occurring, except Silydianin as a diastereomers A and B) with Silybin forming 30-60% of the mixture. Studies suggest Silymarin can protect the Liver against excessive alcohol. The liver metabolises 90% of Alcohol into Acetaldehyde then Acetate generated by ADH (Alcohol Dehydrogenase) and Cytochrome P450. The negative effects of (Ethanol) Alcohol are caused by metabolism of Acetaldehyde. Silymarin may influence alcoholic Liver damage by inhibiting zinc-containing alcohol dehydrogenase. Silymarin Flavonolignins were tested, only 2,3 dehydro derivatives (2,3-dehydroSilybin and 2,3-dehydrosilychristin) significantly Chelated Zinc ions. This was determined by their effects on yeast and equine ADH indicating zinc chelation. All the Silymarin Flavonolignans have an affinity for Zinc ionophoric influx.

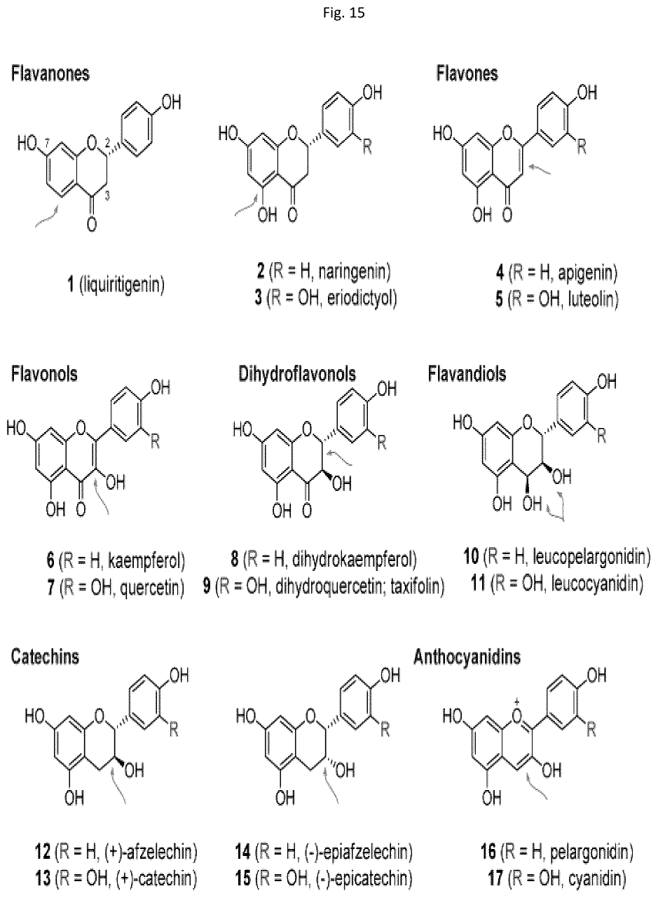

FIG. 15

Flavonoid is derived from Latin Flavus meaning yellow. Flavonoids and stilbenes are derived from a P-Courmaroyl-CoA starter unit extended by the addition of three units of malonyl-CoA. A group of plant enzymes that belong to the family of type 3 Polyketide synthesis (PKS) controls the extension reaction, as well as further modification steps such as cyclisation, enolization, and decarboxylation. The initially formed polyketide may be subsequently folded in two different ways, giving rise to either stilbene (through aldo and ester hydrolysis by stibene synthase) or Nargingenin Calcone (via a claisen-like reaction catalyzed by chalcone synthase in the formation of P-coumaroyleyclohexantrione). Naringenin Chalone can then undergo enolization and a Micheal-type nucleophilic attach of OH-group onto the Alph-Beta-unsaturated ketone (through Chalcone Isomers) forming a general 15-carbon flavonoid structure consisting of two phenyl rings (A and B) and a six-membered heterocyclic Ring. Alternatively the action of Chalcone reductase may generate Isoflavonoids. The primary forms of Flavanone structures (Naringenin) may give rise to a wide range of variants on this basic skeleton, Including Flavons, Flavonols, dihydroflavonols, catechins, and Anthocyanidins. Each of the named subgroups can undergo a different degree of hydroxylation of the Aromatic Rings and these groups can be further modified by methylation, prenylation, glycosylation, or dimethylaallylation. Some flavonoids structures (such as liquiritigenin) may lose one of the hydroxyl groups. Liquiritigenin a plant flavanone, has been found to reduce TC, LDL and Atherogenic Index. Liquiritigenin (Agonist of Estrogen Receptor) also replenishes limited supply of endogenous chain breaking antioxidants such as Alpha tocopherol, by chelating divalent pro-oxidant transition metals like copper, Iron, and Zinc. These compounds Liquiritegenin, Naringenin (Naringin, Eriodictyol), Apigenin, Luteolin, Quercetin (dihydroquercetin), Kaempferol (dihydrokaemferol), Leucoplangonidin (Leucocyanidin), Taxifolin, Myricetin, catechin (Afzelachin), epicatechin (Epiafzelechin), epigallocatechin, Theoflavin, Pycnogenol (procyanidins, Tannins) and Anthocyanins are all zinc chelators and cause zinc ionophoric influx. These have been fully vetted and are effective (aside from Naringenin, Naringin, and Eriodictyol not knowing the percentage of zinc chelation). Rohitukine (is an Alkoloid), Flavopiridol (aka alvocidib) and riviciclib are being vetted successfully for anti-carcinoma effects on humans (they could have some zinc chelating effects also).

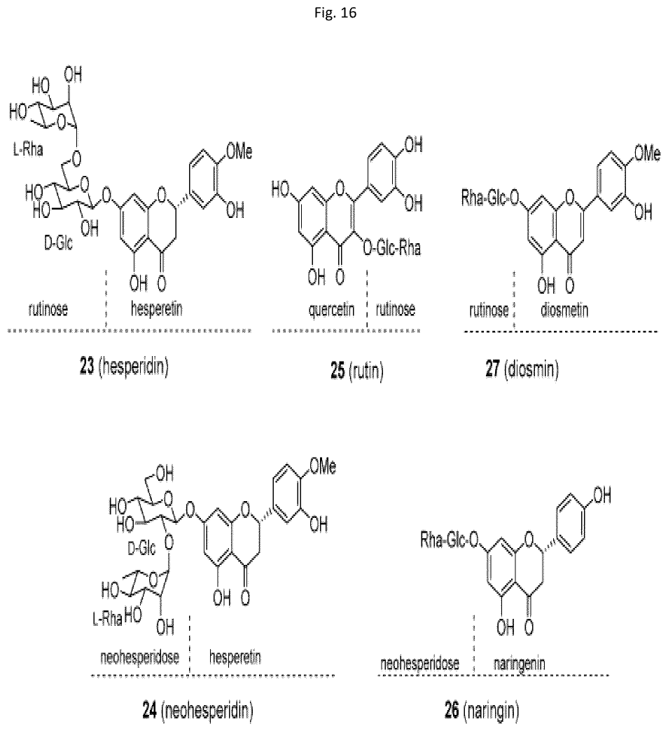

FIG. 16

Rutin, Rue, and Hesperidin (Rutaceae) products are claimed to be of value in the treatment of cardiovascular conditions related to endothelial dysfunction, Post thrombotic syndrome, and venous insufficiency by reducing fragility of blood capillaries and increasing flexibility of blood vessels. Naringin, Naringenin, and hesperidin are the main cause of the drug dose elevating effects (in Grapefruit juice) and are thought necessary for the metabolism of some drugs. This is thought to be accomplished by inhibiting organic anion-transporting, polypeptides, and inhibiting cytochrome P450 enzymes. Neohesperidin-DHC is used as a non-saccharide flavor enhancer, Artificial sweetener. Some Animal studies indicate to use low doses to avoid adverse reactions. Daflon (90% diosmin, Lamiaceae) increases venous tone and resistance in small blood vessels and treats cardiovascular conditions and hemorrhoids. Theaflavins has a strong antiviral potential and reduces Psychophysiological stress. These compounds Rutin (Rutinose), Hesperidin (neohesperidose), Naringin, Naringenin, diosmin (Diosmetin, Daflon), and Quercetin are all zinc chelators and cause zinc ionophoric influx. Quercetin of this list is vetted as a zinc ionophoric influx and the others listed it is unknown the percentage of zinc crossing the cell membrane between 5%-100%.

Spinach contains several important plant compounds and essential amino acids. Querctin, Lutein, Kaempferol, Zeaxanthin, and Nitrates are some of the compounds. Zeaxanthin, meso-zeaxanthin, and Lutein are three compounds found in the macular region of the primate retina and are yellow. Without this macular pigment is inversely associated with increased incidence of macular degeneration. Intake of zeaxanthin, meso-zeaxanthin, and Lutein reduces macular degeneration. Quercetin and Kaempferol are associated with reducing inflammation by inhibiting Interleukin Beta, Interleukin 8, Interleukin 6, and augmenting antioxidant defense against free radicals. Both are excellent zinc chelators and can help zinc cross the cell membrane to stimulate the body's immune system. Spinach contains Nitrates that promote heart health.

Kale is broken down into sulfur-containing compounds glucosinolates broken down into Isothiocyanates and Indole-3-carbinol that inhibit the inflammatory process. Kale (Brassicaceae) also has Flavanoids (glycosylated flavanols) and phenolic compounds Quercetin, Kaempferol, and Isorhamnetin. Phenolic compounds Quercetin, Kaempferol, and Isorhamnetin are responsible for decreasing antioxidants and free radicals by opening and chelating metals (zinc) across the cells plasma membrane. Kale also has three major carotenoids Lutein, Violaxanthin, and Neaxanthin. Kale contains malic acid, ctric acid, (oxalic acid is a zinc chelator that can cause stones) ferulic Acid, Caffeic Acid and Chlorogenic Acid that are zinc ionophoric influx metal chelators (percentage if not listed unknown).

Kale and Spinach contain these Amino Acids that they share Argenine, Histadine, Isoleucine, Leucine, Lycine, Methionone, phenglolanine, threonine, valine, and tryptophan and all happen to be great zinc chelators. This makes them excellent for zinc ionophoric influx (percentage if not listed unknown).

Onions (Allium Cepa) are a wonderful source of antioxidants. Onions have Phytochemicals, Organosulfer compounds, phenolic compounds, polysaccharides, and Saponins. Saponins have an antimicrobial effect. Examples of Phenolic and Organosulfer-containing compounds are Cysteine Sulfides (Smell bad), Quercetin, Quercetin Glucosides (Quercetin-3-Glucoside, Quercetin-4-Glucoside, Quercetin-3-Beta-D-glucoside), Isohamnetin glucosides, Kaempferol glucosides, Onionin A, Cycloalliin, Isoalliin, Methiin, Propiin, and Rutin. Quercetin, Quercetin Glucosides (Quercetin-3-Glucoside, Quercetin-4-Glucoside, Quercetin-3-Beta-D-glucoside), Isohamnetin glucosides, Kaempferol glucosides, Cycloalliin, Isoalliin, Methiin, Propiin, and Rutin are excellent zinc chelators and cause zinc ionophoric influx (percentage if not listed unknown).

Garlic (Allium Sativum) is in the same family and has Quercetin, Quercetin Glucosides (Quercetin-3-Glucoside, Quercetin-4-Glucoside, Quercetin-3-Beta-D-glucoside), Isohamnetin glucosides, Kaempferol glucosides, Cycloalliin, Isoalliin, Methiin, Propiin, and Rutin as excellent zinc chelators and cause zinc ionophoric influx (percentage if not listed unknown).

Cherry Tomatoes (Salanum Lycopersicum) and 5 types of tomatoes have many compounds including citric acid, Alic Acid, Ascorbic Acid (vitamin C is also a zinc chelator), Succinic acid, Naringenin, Naringenin-7-O-glucoside, Naringenin dihexose, Eryiodictol, Quercetin Rutinoside, Quercetin Rutinoside hexodide, Quercetin Rutinoside Pentoside, Kaempferol-Rutinoside pentoside, Kaempferol Rutinoside, Chlorogenic Acid, Dicaffeoyl Quinic Acid, Tricaffeoyl Quinic Acid, Caffeic Acid (abundant), Chlorogenic Acid, P-Cumaric Acid, Ferulic Acid, Gallic Acid, Siringic, Vanellic, Catechin, Quercetin (abundant), Rutin, Lycopen (abundant), Lutein (abundant), Beta carotene, Alpha Carotene, zea-xanthin (stops macular degeneration), Glutamine, Theronine, Isoleucine, Glycine, Tyrosine, Valine, Aspargine, Leucine, Phenylalanine, Tryptophan, Cystein, Glutamic Acid, Serine, Propanoic Acid, Succinate Acid, Malic Acid. These compounds listed for tomatoes have zinc chelating properties and zinc ionophoric influx capabilities. However, they range in chelation and influx capacity anywhere from 5%-100%.

Melatonin is good at reducing oxidative actions of the cells like ROS. Tryptophan is derived from Melatonin. NRF2 is a transcription factor. NRF2 elicits an antioxidant response when enzymes bind to NRF2. Tryptophan reflects the up regulation of NRF2. Tryptophan and melatonin reduce inflammation. However Tryptophan balances the stomach flora and is a precursor to pyruvate that is a part of the Krebs cycle for producing cellular energy. Tryptophan is an ingredient I intend to use for its many versatile actions. Combining tryptophan with other zinc cations is important. Melatonin might also prove to be important as research is developed.

There are an estimated 500,000-600,000 plant derived compounds that have been described, of which nearly 100,000 have promising biological activity. Those 100,000 compounds can be of value for the treatment of diseases. There are many chemical groups such as polyketides, cannaboids, and many other plant based compounds for healing therapeutics. Some examples are Shikimate derived compounds (Tamiflu, etoposide, teniposide), terpanoids (artemesinin and Taxol) and high molecular weight compounds (polysaccharides). The higher clinically and medically used include plant derived alkaloids like Novocain, tubocuanine, morphine, quinine, vincristine, vinblastine, ellipticine, and physostigmine. Alkoloids have found there place in pharmacology while interest in flavonoids is on the rise. Perhaps the most common and frequent are aromatic polyketides that have been seriously studied in recent years.

Polyphenols are described as having a hydroxyl group and one or more phenolic rings for their chemical properties. Polyphenols are listed as first Flavonoids like Anthocynanines, Flavones, Flavonols, isoflavones, and flavan-3ols. I have focused mostly on the flavonoids. Other polyphenols are phenolic acids, polyphenolic acids, polyphenolic amides, polyphenol compounds. I also have focused on some polyphenol compounds as well. Flavonoids have an amazing ability to reduce virility and inflammation. The antiviral properties have interactions between phenyl rings and viral proteins and or RNA that interfere with signaling of Mitogen Activated Protein Kinase. These Flavonoids also decrease pro-inflammatory interleukins like IL-1B, IL-6, IL-8.

I wanted to discuss Isoflavonoids briefly. Isoflavonoids like clovers and lucerne (daidzein, coumestans) have sufficient estrogenic activity that effects reproduction of domesticated or grazing animals. Another term for estrogenic Isoflavonoids is phytoestrogens. Their planar molecule mimics the shape and polarity of the steroid hormone estradiol. These legumes, lucerne, and clovers (daidzein, coumestans) must be restricted from animals. Although, These legumes, lucerne, and clovers (daidzein, coumestans) are not as harmful to humans with the appropriate amounts. However, Flavanones like liquiritigenin and Naringenin can form Isoflavonoids geinstein and daidzen via intermediate hydroxyflavanones. These Isoflavonoids can be changed to Formononetin (largest isoflavonoid amount in Clovers), and then to retenone and pisitan. Retenone and retenoids are used to eradicate undesirable fish and insects. Glycine max Leguminosael/Fabaceae (Soybean) contains Daidzein and geistein and are used by women to decrease estrogen dependent cancers. Daidzein, Geistein, and Formonetin can inhibit Breast and Uterine cancers as well as balance menopause. Another Isoflavanoid function is for Medicarpin (eg. Pterocarpens) lecerne and Pisatan pea (Pisum Sativum) are used for humans as Antifungals. Isoflavonoids are identified by a structural description of hydroxylation and Alkalation reactions varying of oxidation level of heterocyclic ring. Isoflavonoids do have anti-inflammatory properties that I have discussed. However, Isoflavonoids have not been described as having any strong zinc Ionophoric influx Properties.

Flavoligans are also a desirable subject to discuss as Silybum or milk Thistle. Silybum has Anti hepatotoxic properties for protection against Liver damaging agents brought by Industry or otherwise. European medicin Silybin and Isosilybin are used against carbantetrachloride, thioacetamide, paracetamol, Alpha-Amanitin, and Phalloin found in death cap fungus. The action of Silybin is on the cellular membrane of hepatocytes inhibiting absorption of toxins. The phenolic nature acts as antioxidants and scavengers of free radicals that cause keep liver damage from happening from chemicals. Silybin and Isosilybin and many of its derivatives have been described in many studies as having properties of a stronger zinc ionophoric influx.

Ivermectin has good zinc ionophoric influx but is approved by the FDA to prevent heartworm disease and prevent parasites. Ivermectin is part of a family of natural Avermectins from soil Actinomycetes. Avermectins are naturally in the soil and in microbiota of animals. Actinomycetes mediate degradation of complex polymers or enzymes like lignin, chitin, or cellulose. Humans can take Ivermectin as a probiotic for commensal gut microbiota. Ivermectin can be useful to reduce inflammation and stabilize the gut. Prolonged use could destabilize the gut normal flora. Ivermectin can be used as an antiviral by inhibiting Importin alpha/B1 heterodimer. This is how it stops viral replication. However, Ivermectin for human consumption in foods or drinks would be unnecessary due to all the other options with similar and usually better benefits. Benefits like a reduction in inflammation and zinc ionophoric influx.

Palmogranite extract has many flavonoids, hydrolysable tannins, and condensed tannins like—Quercetin, Kaemferol, Luteolin, delphinidin, cyanidin, ellagitannin, punicalagin, gallotannins, and Punicalin. Quercetin, Kaemferol, Luteolin are vetted zinc chelators. Cyanidin, ellagitannin, punicalagin, gallotannins, and Punicalin are also zinc chelators (The percent of Zinc ionophores crossing the cell membrane is unknown, However these are considered strong chelators of zinc). Punicalagin is already a well-known antiviral medicine even used for HIV.

Artemesia Annu (wormwood, Sweet Annu), Qinghoa) is a very interesting plant extract. Artemesia Annu has a very strong zinc ionophoric influx capabilities. Artemesia Annu also has been used by the Chinese to fight covid-19, SARS, and MERS. Artemesia Annu is more effective with Zinc and Phenols (Some use hydrogen peroxide as well to help zinc and calcium cross the plasma membrane). Memorial Sloan and Kettering are using Artemesia Annu as the plant base for their new cancer treatment for colon (prostate and Pancreas as well) cancer. They had a 16 patient trial with colon cancer and all 16 patients had spontaneous remission. They warn not to take the Artemesia Annu because of liver damage (leads to skin rash) and digestive issues. However other studies show very few side effects. This is an amazing product to try. Other names include Artelinate (Orally), Artemisinin (oral or interveneous), and Artesunate. Artemesia Annu is excellent for zinc ionophoric influx.

Hinokitiol (Cupressaceae, Chamaecyparis) is a tropolone natural monoterpenoid and is used for skin care. Hinokitiol is also an iron chelator to suppress cell growth and disrupt Androgen receptors signaling in prostate Cancer and to suppress Teratocarcinoma in F9 cells. Hinokitiol is also useful to Chelate zinc for zinc ionophoric influx to suppress viruses.

### NON PROVISIONAL UTILITY APPLICATION DETAILED DESCRIPTION FORMULAS LISTED TO REFER BACK TO BRIEF DESCRIPTION AND DIAGRAM

### Influx Vitamins

There are many foods with the high values of Quercetin that can replace Elderberry and the capacity to be effective. These foods are any berry extract especially (1) Blueberries, (2) Strawberries, (3) Phytolacca, (4) Acai, just to name a few. Quercetin also has high values in Cherries and grapes. Quercetin also has high values in red onions, Kale, Cherry Tomatoes, and Spinach. The goal is to get enough milligrams through extract of these foods to be effective. This could mean values of 2000-5000 mg of fruit or vegetable per drink or vitamin product. There are other options other than Quercetin or fruit and vegetable extracts. These other options are Fruit and vegetable carbon compounds. These compounds are listed below. These carbon compounds are from the diagrams listed and research.

Amazingly these foods also have other carbon compounds that also have the ability to chelate zinc as well as Quercetin. Quercetin, Dihyroquercetin, Luteolin, Epigallatechin Gallate, Phloritin, Tannic acid, Xanthohumol, Naringenine (needs more testing), and Gallic Acid to name a few are effective in Zinc ionophoric Influx.

Other zinc chelation and zinc ionophoric influx carbon compounds Epigallocatechin, Quercetin, Luteolin, Tannic Acid, Gallic Acid, Caffeic Acid, Catechol, Taxifolin, Rutin, are best for zinc Ionophoric influx. Resveratol, Phloratin, Naringin, and Catechin are strong zinc chelators but weaker zinc ionophoric influx (55-10%).

We will use cherries, blueberries, berries, and grapes.

Elderberry have P-hydroxybenzoic Acid (3.45), Protocatechuic Acid (42.7), P-Coumaric Acid (1.82), Gallic Acid (3.93), CHolorgenic Acid (7.52), Caffeic Acid (3.07), Quinic Acid (310), Quercetin (6.17), Kaempferol-3-o-Glucoside (0.46), Quercetin-3-o-hexide (18), Amentoflavone (0.36), Rutin (5.11), Esculetin (0.27) as carbon compounds. These all have a strong ability to chelate zinc and various mild to strong ability for zinc ionophoric influx. However, only Quercetin, Quercetin-3-O-Hexide, Kaemferol-3-O-Glucoside, and Gallic Acid have a strongest zinc ionophric influx.

Grapes have Quercetin, Kaempferol, myricetin (laricitin is produced from myricetin and syringetin is derived from Laricitin), Laricetin (unknown % zinc influx, inhibits Allergies, opens airway), syringetin (unknown % zinc influx, bone formation) catechin, epicatechin, epicatechin-3-0-gallate, Isorhamnetin, Proanthocyonidins, and Anthocynonidins as strong zinc Chelators. Anthocynonidins in grapes that have strong zinc ionophoric influx are Pelargonidin, cyanidin, Delphinidin, peonidin, petunidin, Malvidin.

Cherries have cyannidin-3-O-glucosyl-rutinside, cyaniding-3-O-rutinoside, Cyanidin-3-O-Monoglucoside, Hydroxy Cinnamic Acid (possibly a good zinc chelator), Chlorogenic Acid, Neochlorogenic Acid, and P-Coumaroylquinic Acid, Catechin, epicatechin, Epigallocatechin, Gallocatechin, Quercetin, Quercetin-3-Rutinose, Apigenin, and Caffeic Acid as compounds to induce zinc ionophoric influx.

Silymarin (Asteraceae, Artichokes) Milk Thistle derivatives are an excellent source for zinc ionophoric influx.

Liquiritegenin, Naringenin (Naringin, Eriodictyol), Apigenin, Luteolin, Quercetin (dihydroquercetin), Kaempferol (dihydrokaemferol), Leucoplangonidin (Leucocyanidin), Taxifolin, Myricetin, catechin (Afzelachin), epicatechin (Epiafzelechin), epigallocatechin, Theoflavin, Pycnogenol (procyanidins, Tannins) and Anthocyanins are all zinc chelators and cause zinc ionophoric influx.

Rutin (Rutinose), Hesperidin (neohesperidose), Naringin, Naringenin, diosmin (Diosmetin, Daflon), and Quercetin are all zinc chelators and cause zinc ionophoric influx.

We will also use red onions, kale, Cherry Tomatoes and spinach.

Spinach contains Querctin, Lutein, Kaempferol, Zeaxanthin, Nitrates, Argenine, Histadine, Isoleucine, Leucine, Lycine, Methionone, phenglolanine, threonine, valine, and tryptophan compounds and all happen to be great zinc chelators and have the ability for zinc ionophoric influx.

Kale contains glucosinolates broken down into Isothiocyanates and Indole-3-carbinol, Quercetin, Kaempferol, Isorhamnetin, Lutein, Violaxanthin, Neaxanthin, malic acid, ctric acid, (oxalic acid is a zinc chelator that can cause stones) ferulic Acid, Caffeic Acid, Chlorogenic Acid, Argenine, Histadine, Isoleucine, Leucine, Lycine, Methionone, phenglolanine, threonine, valine, and tryptophan as compounds that are excellent for zinc ionophoric influx.

Onions have Quercetin, Quercetin Glucosides (Quercetin-3-Glucoside, Quercetin-4-Glucoside, Quercetin-3-Beta-D-glucoside), Isohamnetin glucosides, Kaempferol glucosides, Cycloalliin, Isoalliin, Methiin, Propiin, and Rutin as compounds and are excellent zinc chelators and cause zinc ionophoric influx.

Garlic has Quercetin, Quercetin Glucosides (Quercetin-3-Glucoside, Quercetin-4-Glucoside, Quercetin-3-Beta-D-glucoside), Isohamnetin glucosides, Kaempferol glucosides, Cycloalliin, Isoalliin, Methiin, Propiin, and Rutin as excellent zinc chelators and cause zinc ionophoric influx.

Tomatoes have citric acid, Alic Acid, Ascorbic Acid (vitamin C is also a zinc chelator), Succinic acid, Naringenin, Naringenin-7-O-glucoside, Naringenin dihexose, Eryiodictol, Quercetin Rutinoside, Quercetin Rutinoside hexodide, Quercetin Rutinoside Pentoside, Kaempferol-Rutinoside pentoside, Kaempferol Rutinoside, Chlorogenic Acid, Dicaffeoyl Quinic Acid, Tricaffeoyl Quinic Acid, Caffeic Acid (abundant), Chlorogenic Acid, P-Cumaric Acid, Ferulic Acid, Gallic Acid, Siringic, Vanellic, Catechin, Quercetin (abundant), Rutin, Lycopen (abundant), Lutein (abundant), Beta carotene, Alpha Carotene, zea-xanthin (stops macular degeneration), Glutamine, Theronine, Isoleucine, Glycine, Tyrosine, Valine, Aspargine, Leucine, Phenylalanine, Tryptophan, Cystein, Glutamic Acid, Serine, Propanoic Acid, Succinate Acid, and Malic Acid as compounds and have zinc ionophoric influx capability.

### DETAILED DESCRIPTION FORMULAS LISTED AND REFERRED BACK TO BRIEF DESCRIPTION AND DIAGRAMS

### Influx Vitamins

Formula 1 Basic Immune Support with Reduction of inflammation.
1-1000 mg Quercetin of any variation (500 mg Quercetin is best for this formula).
1-50 mg of zinc of any variation (50 mg of Zinc is best for this formula).
Includes: Quercetin-3-O-Hexide, Quercetin-3-Beta-D-Glucoside, Quercetin-4-Glucoside, Quercetin Rutinside Hexide, Quercetin Rutinside Pentoside (as Esters of Quercetin)

Formula 2 Basic Immune Support with Reduction of inflammation.
1-100 mg Dihydroquercetin (Taxifolin) of any variation (50 mg Dihydroquercetin is best for this formula).
1-50 mg of zinc of any variation (50 mg of Zinc is best for this formula).

Formula 3 Basic Immune Support with Reduction of inflammation.
1-200 mg Luteolin of any variation (100 mg Luteolin is best for this formula).
1-50 mg of zinc of any variation (50 mg of Zinc is best for this formula).

Formula 4 Basic Immune Support with Reduction of inflammation.
1-500 mg Epigallatechin Gaallate of any variation (400 mg Epigallatechin Gallate is best for this formula). (Extract from green tea)
1-50 mg of zinc of any variation (50 mg of Zinc is best for this formula).

Formula 5 Basic Immune Support with Reduction of inflammation.
1-20 mg Phloretin of any variation (5-10 mg Phloretin is best for this formula). (Interact with meds) (Maybe not usable, more studies are needed) (Apple have 5 mg/100 gram of phloretin to extract)
1-50 mg of zinc of any variation (25 mg of Zinc is best for this formula).

Formula 6 Basic Immune Support with Reduction of inflammation.
1-1000 mg Tannic Acid of any variation (750-1000 mg Tannic Acid by using 1500 mg of Galla Chinensis is best for this formula (Galla Chinensis has 50-70% Gallo tannins of its weight (Gallo Tannins are the AKA of Tannins)). (500 mg Black tea at 13 percent is 65 mg of tannic acid) (Quercus Infectoria Galls are a great source of Tannic Acid). (Blueberries have 0.16 mg/gram of tannic Acid)
1-50 mg of zinc of any variation (50 mg of Zinc is best for this formula).

Formula 7 Basic Immune Support with Reduction of inflammation.
1-200 mg Xanthohumol of any variation (150 mg Xanthohumol is best for this formula).
1-50 mg of zinc of any variation (50 mg of Zinc is best for this formula).

Formula 8 Basic Immune Support with Reduction of inflammation.
1-1000 mg Naringenin of any variation (500 mg Naringenin is best for this formula).
Includes: Naringenin-7-o-glucoside, Naringenin-dihexose (Tangerine 10 mg/100 G, source)
1-50 mg of zinc of any variation (50 mg of Zinc is best for this formula).

Formula 9 Basic Immune Support with Reduction of inflammation.
1-500 mg Gallic Acid of any variation (250 mg Gallic Acid by using 1500 mg of Galla Chinensis is best for this formula (Galla Chinensis has 70% Gallo tannins of its weight and 20% of those are Gallic Acid). (Blackberry 5.58 mg/gram is Gallic Acid; Blueberry 9.44 mg/gram is Gallic Acid in extract) (one study used ¼-1 ml Gallic Acid as safe for humans) (In natural form of Fruits or plants 250 mg is safe)
1-50 mg of zinc of any variation (50 mg of Zinc is best for this formula).

Formula 10 Basic Immune Support with Reduction of inflammation.
1-500 mg Caffeic Acid of any variation (10 gram times 2-3 cups of coffee produce 250-500 mg Caffeic Acid is best for this formula). Coffee drinkers will intake caffeic acid up to 2 grams per day from several cups of coffee). 800 mg Green Coffee bean extract is 50% Chlorogenic acid and half of the Chlorogenic Acid is Caffeic Acid (25%) 200 mg. (¼-1 ml Caffeic Acid was considered safe in one study for humans which I suspect is too conservative) Chlorogenic Acid is Ester of Caffeic And Quinic Acid.
1-50 mg of zinc of any variation (50 mg of Zinc is best for this formula).

Formula 11 Basic Immune Support with Reduction of inflammation.
1-200 mg Catechol in the form of Rodiola Rosea 100-200 mg, L-tyrosine 300-600 mg, L-methionine 25-50 mg, Coenzyme Q10 1.5 mg-3 mg of any variation (Catechol is otherwise toxic). (Large quantities not usable, more studies needed) (used in plastics and rubber production)
1-50 mg of zinc of any variation (50 mg of Zinc is best for this formula).

Formula 12 Basic Immune Support with Reduction of inflammation.
1-1000 mg Rutin of any variation (500 mg Rutin is best for this formula). (Quercetin-3-Rutinoside)
1-50 mg of zinc of any variation (50 mg of Zinc is best for this formula).

Formula 13 Basic Immune Support with Reduction of inflammation.
1-1000 mg Resveratol of any variation (700 mg Resveratol is best for this formula).
1-50 mg of zinc of any variation (50 mg of Zinc is best for this formula).

Formula 14 Basic Immune Support with Reduction of inflammation.
1-1000 mg Naringin of any variation (500 mg Naringin is best for this formula).
1-50 mg of zinc of any variation (50 mg of Zinc is best for this formula).

Formula 15 Basic Immune Support with Reduction of inflammation.
1-500 mg Catechin (Afzelachin) of any variation (200 mg Catechin is best for this formula, from green tea)
1-50 mg of zinc of any variation (50 mg of Zinc is best for this formula).

Formula 16 Basic Immune Support with Reduction of inflammation.
10-50 milligrams per kilogram P-hydroxybenzoic Acid (P-hydroxybenzoic Acid used as antimicrobial and antifungal, toothpaste, mountain dew, industrial uses, and packaging. 4-6 grams used daily toxic up to 50 grams daily can be processed by man, not as useful in small amounts)
1-50 mg of zinc of any variation (50 mg of Zinc is best for this formula).

Formula 17 Basic Immune Support with Reduction of inflammation.
10-1000 microgram/liter Protocatechuic Acid of any variation (500 microgram/liter Protocatechuic Acid is best for this formula). Reduces Insulin, modulates gut. (100 mg/kg is considered safe) (Extracts from bitter melon, Black olive, Raspberry, and Ligonberry) (Cherrys 40 mg/100 g and raspberry 10 mg/100 g)
1-50 mg of zinc of any variation (50 mg of Zinc is best for this formula).

Formula 18 Basic Immune Support with Reduction of inflammation.
1-1000 microgram/liter P-Coumaric Acid of any variation (1000 microgram/liter P-Coumaric Acid is best for this formula). Also 8.11-16.22 mg/kg of P-Coumaric Acid is considered safe in some studies. (Humans take 8-27 mg per day as additives). (Extracted from strawberries 29 mg/kg or cinnamon 335 mg/kg)
1-50 mg of zinc of any variation (25 mg of Zinc is best for this formula).

Formula 19 Basic Immune Support with Reduction of inflammation.
1-1000 mg Chlorogenic Acid of any variation (150-500 mg Chlorogenic Acid is best for this formula).
1-50 mg of zinc of any variation (50 mg of Zinc is best for this formula).

Formula 20 Basic Immune Support with Reduction of inflammation.
1-1000 mg Quinic Acid of any variation (25-200 mg Quinnic Acid is best for this formula). Cranberry and quinine 500 mg is 5-6% quinic Acid)
1-50 mg of zinc of any variation (50 mg of Zinc is best for this formula).

Formula 21 Basic Immune Support with Reduction of inflammation.
1-1000 mg Kaempferol of any variation (100-300 mg Kaempferol is best for this formula). (Dihydrokaempferol is an aromadendrin double bonded deriving into kaempferol) (raw Capers 259 mg/100 grams best source)
Includes: Kaempferol-3-O-Glucoside, Kaempferol-Rutinside-Pentose, Kaempferol-Rutinside
1-50 mg of zinc of any variation (50 mg of Zinc is best for this formula).

Formula 22 Basic Immune Support with Reduction of inflammation.
1-1000 mg Amentoflavone of any variation (40-200 mg Amentoflavone is best for this formula). Selaginella Tamariscina extract
1-50 mg of zinc of any variation (50 mg of Zinc is best for this formula).

Formula 23 Basic Immune Support with Reduction of inflammation.
1-1000 mg Rutin of any variation (500 mg Rutin is best for this formula). (Quercetin-3-Rutinoside same as Rutin)
1-50 mg of zinc of any variation (50 mg of Zinc is best for this formula).

Formula 24 Basic Immune Support with Reduction of inflammation.
1-4 g Esculetin of any variation (1.5 g Cortex Fraxini (Esculetin) is best for this formula).
1-50 mg of zinc of any variation (50 mg of Zinc is best for this formula).

Formula 25 Basic Immune Support with Reduction of inflammation.
1-1000 mg L-glutathione of any variation (200 mg L-glutathione is best for this formula).
1-50 mg of zinc of any variation (50 mg of Zinc is best for this formula).

Formula 26 Basic Immune Support with Reduction of inflammation.
1-2000 mg Myricetin of any variation (500-1000 mg Myricetin is best for this formula).
1-50 mg of zinc of any variation (50 mg of Zinc is best for this formula).

Formula 27 Basic Immune Support with Reduction of inflammation.
1-15 mg Laricetin of any variation (10 mg Laricetin is best for this formula).
1-50 mg of zinc of any variation (50 mg of Zinc is best for this formula).

Formula 28 Basic Immune Support with Reduction of inflammation.
1-1000 mg Syringetin (Jumbal Seed) of any variation (450 mg Syringetin in Jumbul Seed (Syzygium Cumini aka Jambolan fruit, Soymida Febrifugal aka Bung fruit sources) is best for this formula).
1-50 mg of zinc of any variation (50 mg of Zinc is best for this formula).

Formula 29 Basic Immune Support with Reduction of inflammation.
1-1000 mg Epicatechin of any variation (500 mg Epicatechin is best for this formula). (extract from Camellia Sinensis 90% Epicatechin) (Epiafzelechin)
1-50 mg of zinc of any variation (50 mg of Zinc is best for this formula).

Formula 30 Basic Immune Support with Reduction of inflammation.
1-1000 mg Epicatechin -3-o-Gallate of any variation (500 mg Epicatechin -3-o-Gallate is best for this formula) (extract from green tea; Camellia Sinensis 90% Epicatechin)
1-50 mg of zinc of any variation (50 mg of Zinc is best for this formula).

Formula 31 Basic Immune Support with Reduction of inflammation.
1-15 mg Isorhamnetin of any variation (5 mg Isorhamnetin is best for this formula). (aka Mythyl Quercetin) (Raw Onions 5 mg/100 grams)
1-50 mg of zinc of any variation (50 mg of Zinc is best for this formula).

Formula 32 Basic Immune Support with Reduction of inflammation.
1-1000 mg Proanthrocynadins (Pinus Pinaster, Pine bark, Pycnogenol) of any variation (6000 mg Pinus Pinaster gives 400 mg Proanthrocynadins is best for this formula).
1-50 mg of zinc of any variation (50 mg of Zinc is best for this formula).

Formula 33 Basic Immune Support with Reduction of inflammation.
1-500 mg Anthocynanidins of any variation (50 mg Anthocyanidins from 250 mg of Bilberry is best for this formula). Anthocynanidins are an Aglycone of Proanthrocynadins. Examples are Malvadi and cynadin.
1-50 mg of zinc of any variation (50 mg of Zinc is best for this formula).

Formula 34 Basic Immune Support with Reduction of inflammation.
1-1000 mg Cyonidin-3-O-Glycosyl-Rutinoside (Tart Cherry 67%) of any variation. (500 mg Tart Cherry) (Cyonidin-3-O-Glycosyl-Rutinoside is made of 67% Prunus Cerasus or Tart Cherry) is best for this formula.
1-50 mg of zinc of any variation (50 mg of Zinc is best for this formula).

Formula 35 Basic Immune Support with Reduction of inflammation.
1-2000 mg Cyonidin-3-O-Rutinoside sweet cherry of any variation (300 mg sweet cherry extract 10/1 ratio: Cyonidin-3-O-Rutinoside 1.4 mg/1 g of sweet cherry is best for this formula) (1.4×3).
1-50 mg of zinc of any variation (50 mg of Zinc is best for this formula).

Formula 36 Basic Immune Support with Reduction of inflammation.
1-1000 mg Cyonidin-3-O-Monoglucooside in blackberry of any variation (300 mg blackberry extract 10/1 ratio: Cyonidin-3-O-Monoglucooside 1.38 mg/1 gram of black berry is best for this formula). Another alternative is 1000 m mg of black bean has 30 mg of Cyan-3-O Glucoside.
1-50 mg of zinc of any variation (50 mg of Zinc is best for this formula).

Formula 37 Basic Immune Support with Reduction of inflammation.
1-1000 mg Hydroxy Cinnamic Acid in cinnamon 0.2 mg/1 gram or olives 0.14 mg/1 gram of any variation (500 mg equals 5000 mg cinnamon extract 10/1 ratio 0.2 mg/1 gram Hydroxy Cinnamic Acid is best for this formula equals 1 milligram of hydroxycinnimic acid). Caffeic Acid, P-Courmaric Acid, Frerulic Acid, and Sinapic Acid are examples of Hydroxy Cinnamic Acid.
1-50 mg of zinc of any variation (50 mg of Zinc is best for this formula).

Formula 38 Basic Immune Support with Reduction of inflammation.
1-4000 mg Neochlorogenic Acid of any variation (4 g plum powdered extract at 0.92 mg/1 gram of Neochlorogenic Acid is best for this formula). (Plum 0.92 mg/1 gram or Chokeberry 0.84 mg/1 gram of Neochlorogenic Acid).
1-50 mg of zinc of any variation (50 mg of Zinc is best for this formula).

Formula 39 Basic Immune Support with Reduction of inflammation.
1-1000 mg Neochlorogenic Acid of any variation (100 mg Apple extract 0.05 mg/1 gram with 10/1 ratio (10000 mg) Neochlorogenic Acid (1 mg) is best for this formula). Neochlorogenic acid is an ester of caffeic Acid and Quinic Acid; and an Isomer of Chlorogenic Acid. (Apple extract 0.05 mg/1 gram or sour cherry 0.014 mg/1 gram Neochlorogenic Acid).
1-50 mg of zinc of any variation (50 mg of Zinc is best for this formula).

Formula 40 Basic Immune Support with Reduction of inflammation.
1-1000 mg Epigallocatechin of any variation (500 mg green tea has 175 mg Epigallocatechin is best for this formula). (Green tea has 25-50% Epigallocatechin)
1-50 mg of zinc of any variation (50 mg of Zinc is best for this formula).

Formula 41 Basic Immune Support with Reduction of inflammation.
1-1000 mg Gallocatechin of any variation (500 mg green tea has 175 mg Gallocatechin is best for this formula). (green tea has 25-50% Gallocatechin)
1-50 mg of zinc of any variation (50 mg of Zinc is best for this formula).

Formula 42 Basic Immune Support with Reduction of inflammation.
1-1000 mg Apigenin of any variation (300 mg Apigenin is best for this formula). (Extract from Matricaria Chamomille or Citrus Aurantium aka bitter orange)
1-50 mg of zinc of any variation (50 mg of Zinc is best for this formula).

Formula 43 Basic Immune Support with Reduction of inflammation.
1-1000 mg Silymarin (milk Thistle; Silybum Marianum) of any variation (400 mg Silymarin is best for this formula). (500 mg of milk Thistle; Silybum Marianum 80% Silymarin)
1-50 mg of zinc of any variation (50 mg of Zinc is best for this formula).

Formula 44 Basic Immune Support with Reduction of inflammation
1-1000 mg Epigallocatechin-3-O-Gallate of any variation (500 mg green tea is best for this formula). (500 mg Extract green tea; Camella Sinesis Leaf 50% mg Epigallocatechin Gallate is 250 mg)
1-50 mg of zinc of any variation (50 mg of Zinc is best for this formula).

Formula 45 Basic Immune Support with Reduction of inflammation.
1-1000 mg Liquiritegenin of any variation (500 mg Glycyrrhiza Glabra (Licorice) 5.5-15.3% of Liquiritegenin yields 40-60 mg is best for this formula).
1-50 mg of zinc of any variation (50 mg of Zinc is best for this formula).

Formula 46 Basic Immune Support with Reduction of inflammation.
1-1000 mg Delphinadin (Raw Bilberry, European Black Currants, Blackberry Juice Concentrate) of any variation (1000 mg Raw Bilberry 250 mg 4/1 ratio has 97.5 mg Delphinadin/1 gram Bilberry is best for this formula) (Black currant extract 100 mg 10/1 ratio has 87.6 mg Delphinadin/1 gram black currant).
1-50 mg of zinc of any variation (50 mg of Zinc is best for this formula).

Formula 47 Basic Immune Support with Reduction of inflammation.
1-1000 mg Peonidin of any variation (500 mg Cranberry 50/1 ratio is 25000 mg cranberry with 40 mg/100 grams of Peonidin is best for this formula). (1000 mg Peonidin) (Cranberry, Bilberry)
1-50 mg of zinc of any variation (50 mg of Zinc is best for this formula).

Formula 48 Basic Immune Support with Reduction of inflammation.
1-1000 mg Petunidin (Bilberry, Blueberry) of any variation (750 mg Bilberry 4/1 ratio is 3000 mg Bilberry with 42 mg/100 g of Petunidin is best for this formula). (126 mg Petunidin)
1-50 mg of zinc of any variation (50 mg of Zinc is best for this formula).

Formula 49 Basic Immune Support with Reduction of inflammation.
1-1000 mg Malvidin (Blueberry) of any variation (300 mg Blueberry 50/1 ratio is 15000 mg Blueberry with 59.6 mg/100 gram of Malvidin is best for this formula). (885 mg of Malvidin)
1-50 mg of zinc of any variation (50 mg of Zinc is best for this formula).

Formula 50 Basic Immune Support with Reduction of inflammation.
1-1000 mg Leucopelargonidin (derivative of dihydrokaempferol) (Kashew nut) of any variation (500 mg Kashew nut, has trace Leukopelargonidin less than 1 mg (more studies needed), is best for this formula).
1-50 mg of zinc of any variation (50 mg of Zinc is best for this formula).

Formula 51 Basic Immune Support with Reduction of inflammation.
1-1000 mg) Leukocyonidin (derivative of dihydroquercetin) of any variation (500 mg Baby Arecanut, has 200 mg of Leucocyonidin, is best for this formula).
1-50 mg of zinc of any variation (50 mg of Zinc is best for this formula).

Formula 52 Basic Immune Support with Reduction of inflammation.
1-1000 mg Theaflavin (Black Tea) of any variation (500 mg Black tea extract is 25% or 125 mg of Theaflavin is best for this formula).
1-50 mg of zinc of any variation (50 mg of Zinc is best for this formula).

Formula 53 Basic Immune Support with Reduction of inflammation.
1-1000 mg Hesperidin of any variation (500 mg Hesperidin is best for this formula). (extract from Citrus Aurantium 90% or pure concentration from Citrus Aurantium) (Neohesperidose AKA)
1-50 mg of zinc of any variation (50 mg of Zinc is best for this formula).

Formula 54 Basic Immune Support with Reduction of inflammation.
1-1000 mg Diosmin of any variation (500 mg Diosmin is best for this formula). (AKA Diosmetin, Daflon) (Extract from Citrus Aurantium or from Citrus Sinensis 90-95%)
1-50 mg of zinc of any variation (50 mg of Zinc is best for this formula).

Formula 55 Basic Immune Support with Reduction of inflammation.
1-500 mg Lutein of any variation (40 mg Lutein is best for this formula). (Extracted from marigold pedal extract, AKA Tagetes erecta)
1-50 mg of zinc of any variation (50 mg of Zinc is best for this formula).

Formula 56 Basic Immune Support with Reduction of inflammation.
1-100 mg Zeaxanthin of any variation (4 mg Zeaxanthin is best for this formula). (Extracted from marigold pedal extract, AKA Tagetes erecta)
1-50 mg of zinc of any variation (50 mg of Zinc is best for this formula).

Formula 57 Basic Immune Support with Reduction of inflammation.
1-3000 mg Argenine of any variation (1000 mg Argenine is best for this formula). (Arginine is extracted from gelatin, soy, red meat, Fish, and whole grain)
1-50 mg of zinc of any variation (50 mg of Zinc is best for this formula).

Formula 58 Basic Immune Support with Reduction of inflammation.
1-1000 mg Histadin of any variation (500 mg Histadin is best for this formula). (Histidine is extracted from gelatin, soy, red meat, Fish, nuts, seeds, eggs, dairy, and whole grain)
1-50 mg of zinc of any variation (50 mg of Zinc is best for this formula).

Formula 59 Basic Immune Support with Reduction of inflammation.
1-3000 mg Isoleucine of any variation (1000 mg Isoleucine is best for this formula). (Isoleucine is extracted by protein hydrolysis of eggs, soy protein, seaweed, turkey, fish, and dairy products. Isoleucine is also produced largely by microbial fermentation.)
1-50 mg of zinc of any variation (50 mg of Zinc is best for this formula).

Formula 60 Basic Immune Support with Reduction of inflammation.
1-2000 mg Leucine of any variation (1000 mg Leucine is best for this formula). (Leucine is produced largely by microbial fermentation. Leucine is extracted from meats, dairy products, soy products, beans, and legumes)
1-50 mg of zinc of any variation (50 mg of Zinc is best for this formula).

Formula 61 Basic Immune Support with Reduction of inflammation.
1-1000 mg Lycine of any variation (500 mg Lycine is best for this formula).
1-50 mg of zinc of any variation (50 mg of Zinc is best for this formula).

Formula 62 Basic Immune Support with Reduction of inflammation.
1-1000 mg Methionine (L-Mehtionine and D Mehtionine) of any variation (500 mg Mehtionine is best for this formula). (Mehtionine is extracted by protein hydrolysis of eggs, soy protein, beans, turkey, fish, and dairy products. Methionine is also produced largely by microbial fermentation. Metbionine may be produced by chemical synthesis)
1-50 mg of zinc of any variation (50 mg of Zinc is best for this formula).

Formula 63 Basic Immune Support with Reduction of inflammation.
1-1000 mg Phenylalanine (L-Phenylalanine) of any variation (500 mg Phenylalanine is best for this formula). (Phenylalanine is extracted by protein hydrolysis of eggs, soy, meat, poultry, fish, beans, and dairy products. Phenylalanine is also produced largely by microbial fermentation.
1-50 mg of zinc of any variation (50 mg of Zinc is best for this formula).

Formula 64 Basic Immune Support with Reduction of inflammation.
1-1000 mg Threonine of any variation (500 mg Threonine is best for this formula). Threonine is extracted by protein hydrolysis of eggs, soy, meat, poultry, fish, beans, and dairy products. Threonine is also produced largely by microbial fermentation or Liquor fermentation.
1-50 mg of zinc of any variation (50 mg of Zinc is best for this formula).

Formula 65 Basic Immune Support with Reduction of inflammation.
1-2000 mg Valine of any variation (1000 mg Valine is best for this formula). Valine is extracted by protein hydrolysis of eggs, soy, meat, poultry, fish, beans, nuts, grain, and dairy products. Valine is also produced largely by microbial fermentation.
1-50 mg of zinc of any variation (50 mg of Zinc is best for this formula).

Formula 66 Basic Immune Support with Reduction of inflammation.
1-1500 mg Tryptophan of any variation (500 mg Tryptophan is best for this formula).Tryptophan is extracted by protein hydrolysis of eggs, soy, meat, poultry, fish, beans, nuts, grain, and dairy products (Griffonia Simplicifolia). Tryptophan is also produced largely by microbial fermentation.
1-50 mg of zinc of any variation (50 mg of Zinc is best for this formula).

Formula 67 Basic Immune Support with Reduction of inflammation.
1-1000 mg Cycloalliin of any variation (1000 mg Garlic (Allum Sativum) yields 10-20 mg of Cycloalliin is best for this formula).
1-50 mg of zinc of any variation (50 mg of Zinc is best for this formula).

Formula 68 Basic Immune Support with Reduction of inflammation.
1-1000 mg Allicin of any variation (1000 mg Garlic (Allum Sativum) yields 10-20 mg of Allicin is best for this formula). Includes: Alliin, IsoAlliin
1-50 mg of zinc of any variation (50 mg of Zinc is best for this formula).

Formula 69 Basic Immune Support with Reduction of inflammation.
1-1000 mg Methiin of any variation (1000 mg Garlic (Allum Sativum) yields 5-20 mg of Methiin is best for this formula).
1-50 mg of zinc of any variation (50 mg of Zinc is best for this formula).

Formula 70 Basic Immune Support with Reduction of inflammation.
1-1000 mg Propiin of any variation (1000 mg Garlic (Allum Sativum) yields 5-20 mg of Propiin is best for this formula).
1-50 mg of zinc of any variation (50 mg of Zinc is best for this formula).

Formula 71 Basic Immune Support with Reduction of inflammation.
1-1000 mg Citric Acid of any variation (500 mg Citric Acid is best for this formula).
1-50 mg of zinc of any variation (50 mg of Zinc is best for this formula).

Formula 72 Basic Immune Support with Reduction of inflammation.
1-1000 mg Allic Acid of any variation (500 mg Allic Acid is best for this formula).
1-50 mg of zinc of any variation (50 mg of Zinc is best for this formula).

Formula 73 Basic Immune Support with Reduction of inflammation.
1-1000 mg Ascorbic Acid of any variation (500 mg Ascorbic Acid (Vit. C) is best for this formula).
1-50 mg of zinc of any variation (50 mg of Zinc is best for this formula).

Formula 74 Basic Immune Support with Reduction of inflammation.
1-1000 mg Succinate Acid of any variation (500 mg Succinate Acid is best for this formula).
1-50 mg of zinc of any variation (50 mg of Zinc is best for this formula).

Formula 75 Basic Immune Support with Reduction of inflammation.
1-1000 mg Eriodictyol of any variation (500 mg Yerba Santa sage for 12-80 mg/gram of Eriodictyol is best for this formula).
1-50 mg of zinc of any variation (50 mg of Zinc is best for this formula).

Formula 76 Basic Immune Support with Reduction of inflammation.
1-1000 mg Dicaffeoyl Quinic Acid of any variation (800 mg green coffee bean extract yields 8.5 mg/gram of Dicaffeoyl Quinic Acid is best for this formula). Includes: or Tricaffeoyl Quinic Acid
1-50 mg of zinc of any variation (50 mg of Zinc is best for this formula).

Formula 77 Basic Immune Support with Reduction of inflammation.
1-1000 mg Ferulic Acid of any variation (500 mg Ferulic Acid is best for this formula). (Extracted from wheat 0.72 mg/1 gram) (called Trans Ferulic Acid)
1-50 mg of zinc of any variation (50 mg of Zinc is best for this formula).

Formula 78 Basic Immune Support with Reduction of inflammation.
1-1000 mg Syringic of any variation (500 mg Syringic is best for this formula). (Extracted from walnuts 451 mg/100 gram or 4.51 mg/1 gram)
1-50 mg of zinc of any variation (50 mg of Zinc is best for this formula).

Formula 79 Basic Immune Support with Reduction of inflammation.
1-1000 mg Vanellic Acid of any variation (300 mg Basil extract 20/1 ratio is 6000 mg Basil: Basil has 14 mg/100 gram of Vanellic Acid is best for this formula). (Ocimum Tenuiform) 840 mg VA
1-50 mg of zinc of any variation (50 mg of Zinc is best for this formula).

Formula 80 Basic Immune Support with Reduction of inflammation.
1-1000 mg Lycopen of any variation (50 mg Lycopen is best for this formula). (Extracted from tomato oleoresin at 6.8% Solonum Lycopersicum)
1-50 mg of zinc of any variation (50 mg of Zinc is best for this formula).

Formula 81 Basic Immune Support with Reduction of inflammation.
1-50 mg Beta Carotene (Vit. A) of any variation (3.5 mg Beta Carotene is best for this formula). (Extracted from Carrots or sweet potato at 9.2-9.4 mg/100 gram) excess is carcinogenic
Includes: Alpha Cerotene although different
1-50 mg Alpha Carotene of any variation (1-6.2 mg Alpha Carotene is best for this formula). (Extracted from Carrots at 6.2 mg/100 gram)
1-50 mg of zinc of any variation (50 mg of Zinc is best for this formula).

Formula 82 Basic Immune Support with Reduction of inflammation.
1-5000 mg Glutamine of any variation (2000 mg Glutamine is best for this formula). (Extract 40 mg/1 gram of fish) (L-glutamine AKA)
1-50 mg of zinc of any variation (50 mg of Zinc is best for this formula).

Formula 83 Basic Immune Support with Reduction of inflammation.
1-1000 mg Threonine of any variation (500 mg Threonine is best for this formula). (Found in beef, poultry, fish, and dairy) (L-Threonine AKA)
1-50 mg of zinc of any variation (50 mg of Zinc is best for this formula).

Formula 84 Basic Immune Support with Reduction of inflammation.
1-3000 mg Glycine of any variation (1000 mg Glycine is best for this formula). (Found in gelatin, beef, poultry, fish, and dairy). (Main ingredient in Collagen) (L-Glycine AKA)
1-50 mg of zinc of any variation (50 mg of Zinc is best for this formula).

Formula 85 Basic Immune Support with Reduction of inflammation.
1-1000 mg Tyrosine of any variation (750 mg Tyrosine is best for this formula). (Found in beef, poultry, fish, sesame seed, whole grains, and dairy) (AKA L-Tyrosine) (precurser brain neurotransmitters)
1-50 mg of zinc of any variation (50 mg of Zinc is best for this formula).

Formula 86 Basic Immune Support with Reduction of inflammation.
1-1000 mg Asparagine of any variation (500 mg Asparagine is best for this formula). (Extracted from Asparagus and Licorice root) (Asparagus 4/1 ratio 400 mg is 1600 mg daily)
1-50 mg of zinc of any variation (50 mg of Zinc is best for this formula).

Formula 87 Basic Immune Support with Reduction of inflammation.
1-2000 mg Cysteine of any variation (1000 mg Cysteine is best for this formula). (Found in beef, poultry, fish, sesame seed, whole grains, hair, and dairy) (AKA L-Cysteine or N-Acytl-Cysteine) (used for respiratory illness or COPD)
1-50 mg of zinc of any variation (50 mg of Zinc is best for this formula).

Formula 88 Basic Immune Support with Reduction of inflammation.
1-4000 mg Glutamic Acid of any variation (1000 mg Glutamic Acid is best for this formula). (Found in beef, poultry, fish, Tomato, whole grains, yeast, and dairy) (Digestion and Brain function) (AKA L-Glutamic Acid)
1-50 mg of zinc of any variation (50 mg of Zinc is best for this formula).

Formula 89 Basic Immune Support with Reduction of inflammation.
1-2000 mg Serine of any variation (1000 mg Serine is best for this formula). (Found in beef, poultry, fish, Nuts (walnuts, peanuts), beans, whole grains, and dairy) (AKA L-Serine) (suppresses dementia and retardation; increases brain function)
1-50 mg of zinc of any variation (50 mg of Zinc is best for this formula).

Formula 90 Basic Immune Support with Reduction of inflammation.
1-1000 mg Propanoic Acid of any variation (500 mg Propanoic Acid is best for this formula). (AKA Propionic Acid) (extracted from bacterial fermentation, dairy, or whole grains) (considered safe in small amounts; larger amounts category 1 toxicity. Studies are being made to add it to supplemental diet) (Now only few countries have 500 mg supplements available) (U.S. uses 1-10 mg as preservative and flavor)
1-50 mg of zinc of any variation (50 mg of Zinc is best for this formula).

Formula 91 Basic Immune Support with Reduction of inflammation.
1-100 mg Succinate Acid of any variation (1 mg Succinate Acid is best for this formula) (Found in beef, poultry, fish, Tomato, whole grains, yeast, and dairy) (considered safe in small amounts found naturally in food; larger amounts category 1 toxicity) (U.S. uses 1-10 mg as preservative and flavor) (not going to be used as major supplement)
1-50 mg of zinc of any variation (50 mg of Zinc is best for this formula).

Formula 92 Basic Immune Support with Reduction of inflammation.
1-2500 mg Malic Acid of any variation (1000 mg Malic Acid is best for this formula). (Extracted from apples, grapes, berries, bananas, and tomatoes) (apples are 7-8 mg/gram on average)
1-50 mg of zinc of any variation (50 mg of Zinc is best for this formula).

Formula 93 Basic Immune Support with Reduction of inflammation.
1-2000 mg Bromelain of any variation (500 mg Bromelain is best for this formula). (Extracted from pineapple and pineapple stem)
1-50 mg of zinc of any variation (50 mg of Zinc is best for this formula).

These vitamin drink formulas and vitamin combinations are superior for immune support and in reducing inflammation.

These vitamin and drink formulas are superior by working in humans by reducing inflammation due to the inhibition of the TWIK2 Channels (potassium) in the Macrophages (White Blood Cells); this inhibits Nlrp3 inflammasome and then in turn inhibits Interleukin Beta signal with in the CNS (central nervous system) that stimulates inflammation.

These vitamins and drink formulas work to further reduce inflammation this happens in the lungs due to the inhibition of TAS2R channels; this inhibits IGE immunoglobulin which reduces allergic inflammation.

My vitamin formulas, gummies, and drink formulas are superior in there combinations to what is out there in the markets; this is due to proper Ingredients for cellular changes and higher milligram.

These vitamins and drink formulas work to further increase the Immune support by using Quinine to inhibit Sars-Covid-2 because of its antiviral activity; this is illustrated in the stomach by inhibiting TMPRSS2 a protease in the colon; sited “Quinine inhibits infection of Human Cell Lines with Sars-Cov-2”. Quercetin, Elderberry, and compounds 1-93 are active bioflavonoids in fruit and vegetable extract also improve digestion.

There is an increase in white blood cell and nerve cell proliferation that increases immune support. Vitamin D is an Autocrine and Paracrine Proliferation (Making these cells) of B cells, T cells, Macrophages (these first three are White Blood Cells), and Dendrites (nerve cells); these cells can interact with Vitamin D and (Bind) Synthesize 125-Dihydroxy Vitamin D; this causes an increase in white blood cell and nerve cell proliferation that increases immune support.

Vitamin D makes Calcium and Bone formation; this increases immune support. Vitamin D makes Collagen matrix mineralization of bone; vitamin D is also Skin Synthesized then sent inactive to the Liver; from there D is sent from the Liver to the Kidney as Active vitamin D; bone formation is important in the long bones for the production of white and red blood cells.

Vitamin C has antiviral properties that reduces inflammation. Vitamin C increases lymphocyte activity, interferon (cell communication) production, modulating Cytokines, endothelial function, reduces inflammation, and storing mitochondrial function.

Zinc when taken regularly inhibits covid-19 replication, especially, when taken with zinc chelator.

Zinc inhibits viral replication by further comprising Zinc Ionophores such as Quinine, Elderberry, Formulas 1-93 primary ingredient organic compounds such as Kaempferol, Epigallatechin Gallate, Isorhamnetin, Proanthocyonidins, Amentoflavone, Apigenin, Methionone, Quinnic Acid, Silymarin, and Quercetin cause an influx of Zinc into human cells; this inhibits viral replication. This is done with plant polyphenols such as flavonoids like Quercetin that act like antioxidants and carry the zinc Ionophore across the cell membrane; Quercetin and compounds 1-93 are flavonoids in elderberry, berries, grapes, Kale, Red onions and other fruits and vegetables.

Vitamin C Recycles Quercetin and Glutathione Bioflavonoid in the body. Essentially doubling the Quercetin. Quercetin inhibits Polymorases, Proteases Reverse Transcriptase, Suppression DNA Gyrase, and binding Viral Capsid Proteins: this recycling of Quercetin and Glutathione inhibits viral replication.

These vitamin and drink formulas are superior by reducing ROS. The organic compounds found in formulas 1-93 can actively reduce ROS Reactive Oxygen Species within human cells, reducing inflammation. Excessive Oxygen within cells causes cell stress and cell Apoptosis.

These vitamins and drink formulas work to further reduce inflammation with the organic compounds found in formulas 1-93 that reduce free radicals and carcinogens, thereby reducing inflammation and giving superior Immune Support.

All formulas are meant to be drink formulas and or vitamin formulas. All Formulas that are meant to be Drink Formulas can be plugged in replacement of Quercetin, Elderberry, or Quinine. Examples of these formulas that these formulas can replace quercetin are in the page 1 field of invention or summary example. Other such interchanges between These Formulas and replacing Quercetin, Elderberry, and Quinine (as well as other Fruits and Vegetables) are in the first Patent. The First Patent is also dealing with Zinc Ionophoric Influx. This is a continuation of that work designed for an international PCT patent.

The basic patent is for Super Vitamin Grouping for reducing inflammation and stimulating the body's immune system as well as reducing arthritis. There are several ingredients as listed below for the perfect blend I have recommended for my patients:
1-1000 mg Quinine of any variation (250 mg or 500 mg Quinine used for these formulas).
1-50 mg of Zinc of any variation (25 mg or 50 mg Zinc used for these formulas).
1-50 mcg 2000 lu D3 of any variation (25 mcg or 50 mcg 2000 IU D3 used for these formulas).
1-2000 Calcium of any variation (200 mg or 400 mg Calcium used for these formulas).
1-500 mg Glucosamine of any variation (500 mg Glucosamine is best for this formula).
1-1000 mg Chondroitin of any variation (150 mg Chondroitin is best for this formula).
1-1000 mg Quercetin of any variation (250 mg or 500 mg Quercetin used for these formulas).
1-2000 mg Elderberry of any variation (500 mg or 1000 mg Elderberry used for these formulas).
1-500 mg of Magnesium of any variation (100 mg Magnesium used for these formulas).
1 mg-1000 mg of Vitamin C (200 mg of Vitamin C used in these formulas).
Monopotassium Phosphate, Potassium Benzoate, and Salt (Sea salt Preferable) used in these formulas

These ingredients can be combined 2-3 or even several formula ingredients together at a time with zinc interchangeably.

Copper should be incorporated in each Formula because all formulas are all dealing with zinc. Copper has the ability when consumed to protect the liver while taking larger amounts of zinc. The amount of copper should be 3 mg or 6 mg per formula.

If a person is having kidney pain due to poison, stones, injury, or failure Dandelion Root is highly recommended for all such issues. Dandelion Extract 1500 mg 2-3 daily is recommended for such issues. Dandelion is composed of Sequiterpene, taraxerol, Chlorogenic Acid, and Chicory Acid as Compounds.

People with PKU (Phenylketonuria) cannot have an abundance of Amino Acids nor take supplements with Amino Acids. Diabetics can be advised by doctors in cases not to take AA.

Zinc Ionophoric influx and zinc chelation is a technique used by doctors and scientists to treat for poisoning.

Zinc is best used in these formulas as 12 mg, 25 mg, as well as 50 mg.

### Summary

My objectives are to patent products with enough volume of mg vitamins to be effective. Also, have perfect recommendation instructions on the container to perfectly allow each consumer to attain quality health. Each product formula has the precise vitamin ingredients for the desired reduction of inflammation and immune support. The main idea is to get fruit and vegetable concentrate extract (powder concentrate extract, syrup concentrate extract, fluid concentrate extract) with high volume of Quercetin or other flavonoids (3 carbon chain) to open up the human cell plasma membrane for a zinc ionophoric influx reaction. Then add sufficient Zinc to enhance the patient's immune system. This was not in the markets at all when I submitted my patent in 2021.

Example: Formula 5 Quercetin, Zinc, D3, Calcium, Sodium, and Potassium Immune Drink
1-5000 mg Quercetin in any variation in a drink form including Quercetin Syrup Diluted with fluid to make an Immune drink or refreshment. 250 mg used in this formula
1-200 mg of Zinc in any variation. 25 mg used in this formula
1-200 mcg of vitamin D3 of any variation. 25 mcg used in this formula
1-2000 mg of Calcium of any variation. 200 mg used in this formula
1-500 mg of Sodium Ions such as in an energy drink (160 mg per 16 oz. is commonly used and is what I use)
1-300 mg of Potassium Ions such as in energy drinks (45 mg per 16 oz. is commonly used and is used)
16-32 oz container is best

This Formula is perfect for Immune Support and Reducing Inflammation. There are no other formulas with this much Quercetin, Zinc, D3, Calcium, and Ions like Potassium and Sodium. The idea is to recommend the consumer drink 2-4 (16 oz.) drinks per day for best results. I am a practicing physician who has not only recommended these same vitamin ingredients to 2000 patients. I have also monitored there progress along with other health recommendations. My patients received exercise, proper diet, rest and spinal manipulations. My patients and I have achieved amazing results in an inhospitable environment such as we have today. However, I must first patent my results of my many healthy patients.

1. A method of inducing and increasing an immune response and reducing inflammation with zinc ionophoric influx by chelating zinc with three carbon compound structures that has two aromatic rings found in fruits and vegetables to open up human cells for zinc ionophores to enter the human cells as well as linear sulfuric structures and other plant compounds and plants that also open up human cells phospholipid bilayer for zinc ionophores to enter.

2. A method of inducing and increasing an immune response and reducing inflammation in addition to chelating zinc with zinc ionophoric influx with other vitamins that are known to stimulate and enhance the immune system, reduce inflammation, and facilitate most of the systems of the body by using Vitamin D3, Vitamin C, Copper, Calcium, Magnesium, Potassium, Sodium, Glucosamine, and Chondroitin.
